(19) United States
(12) Patent Application Publication
(10) Pub. No.: US 2022/0195378 A1
(43) Pub. Date: Jun. 23, 2022

(54) METHOD FOR PREPARING A BIOMASS OF STABLE FREEZE-DRIED BACTERIAL CELLS AND DETERMINING THE STABILITY THEREOF BY MEANS OF A CYTOFLUOROMETRY METHOD

(71) Applicant: PROBIOTICAL S.P.A., Novar (NO) (IT)

(72) Inventors: Vera MOGNA, Novara (NO) (IT); Marco PANE, Novara (NO) (IT); Serena ALLESINA, Novara (NO) (IT)

(21) Appl. No.: 17/604,345

(22) PCT Filed: Apr. 20, 2020
(86) PCT No.: PCT/IB2020/053732

§ 371 Date: Oct. 15, 2021

(30) Foreign Application Priority Data
Apr. 18, 2019 (IT) 102019000006056

(51) Int. Cl. C12N 1/04; C12N 1/20; C12Q 1/06; A23L 33/135

(52) U.S. Cl. CPC C12N 1/04; C12N 1/20; A23V 2002/00; A23L 33/135; C12Q 1/06

(57) ABSTRACT

A biomass of freeze-dried bacterial cells and related devices, compositions and method of preparation are described. The method comprises (i) fermenting a previously prepared biomass of bacterial cells (bacterial biomass) to obtain a biomass of fermented bacterial cells (fermented biomass); (ii) concentrating the fermented biomass obtained from step (i) up to obtaining a biomass of concentrated bacterial cells (concentrated biomass) having a bacterial cell concentration comprised from 1×10⁶ cells/ml of liquid biomass to 1×10¹² cells/ml of liquid biomass; (iii) mixing the concentrated biomass obtained from step (ii) with a solution comprising or, alternatively, consisting of: (a) at least one phosphorous salt, and (b) at least one polyhydroxy substance to obtain a cryoprotected biomass of bacterial cells (cryoprotected biomass); (iv) freeze-drying the cryoprotected biomass obtained from step (iii) to obtain a biomass of freeze-dried bacterial cells (freeze-dried biomass).

### FIELD OF THE INVENTION

The present invention regards a biomass of freeze-dried, high-concentration and stable bacterial cells. Furthermore, the present invention regards a method for preparing said biomass of freeze-dried, high-concentration and stable bacterial cells. The freeze-dried bacterial cells of the present invention have a stability in terms of viability expressed in AFU, determined by means of a cytofluorometry method, greater than the stability determined on the same cells by means of plate count and expressed in CFU. Lastly, the present invention regards a pharmaceutical composition, or a medical device composition, or a cosmetic use composition, or a food supplement composition, or a food for special medical purposes (FSMP) composition (all of these compositions referred to, for the sake of brevity, as the “compositions of the present invention”) comprising, said compositions, said biomass of freeze-dried, high-concentration and stable bacterial cells.

### BACKGROUND OF THE INVENTION

In recent years, products containing bacterial cells are gaining increasing market shares both in the food industry (for example for the production of dairy products), in the food supplements industry (for example probiotic products), and in the pharmaceutical industry such as Live Biotherapeutic Products (LBP). In these industrial sectors, and with specific reference to this type of products, the aspects related with the stability and viability of the bacterial cells are of extreme importance. The stability in terms of viability and integrity of the bacterial cells strongly depends on the method used to produce them. As a matter of fact, the micro-organisms or bacterial cells contained in said products are very sensitive to the process conditions and parameters for their production and they are also very affected by the environmental preservation conditions, in particular the bacterial cells are sensitive and are affected by temperature, light, UV rays, oxygen, activity water, humidity of the production environment and preservation downstream of the production process. Furthermore, most micro-organisms are anaerobes or, however, extremely sensitive to exposure to oxygen due to the generation of oxygen free radicals that reduce the viability thereof.

Therefore, such circumstance represents one of the main limits in the distribution of biomasses of bacterial cells in certain geographical areas (by way of example, in zones IV.A and IV.B. identified by the World Health Organization), in which it is extremely difficult to ensure conditions that guarantee a viability of a sufficiently high number of micro-organisms or bacterial cells, and for sufficiently long periods of time, to still have significant efficacy when they are used or consumed.

In October 2005, the WHO recommended dividing climatic zone IV into two different zones, introducing zone IV.A (hot and humid) and zone IV.B. (hot and very humid). So today there are 5 different climatic zones and 5 different conditions for conducting stability studies, to be used depending on the target market
ZONE I: Temperate climate—Long-term storage conditions: 21° C./45% R.H.
ZONE II: Subtropical and Mediterranean climate—Long-term storage conditions: 25° C./60% R.H.
ZONE III: Hot and dry climate—Long-term storage conditions: 30° C./35% R.H.
ZONE IV.A: Hot and humid climate—Long-term storage conditions: 30° C./65% R.H.
ZONE IV.B.: Hot and very humid climate—Long-term storage conditions: 30° C./75% R.H.

In order to facilitate the knowledge of the conditions required for the conduction of studies in the different counties, the WHO published a list of the acceding States, with the relevant long-term storage condition in the WHO Technical report series No 953, 2009 Annex 2 “Stability testing of active pharmaceutical ingredients and finished pharmaceutical products” guideline.

US 2004043374 refers to the preservation and stability of biological samples by using techniques such as freezing and freeze-drying. The described protection solutions are prepared using aqueous solutions in phosphate buffer, and by adding predetermined amounts of a polyhydroxy substance and phosphate ions. These buffered protection solutions are mixed with the biological material at amounts depending on the type of biological material selected. However, this document neither describes the use of pyrophosphate nor evaluates the advantages resulting from the use thereof in a cryoprotection solution. Furthermore, the protection solution used is buffered, and the buffer is preferably a phosphate buffer. As a result, the phosphate ions present in the solution mixed with the biomass are, at least partly, derived from the buffer solution.

WO20147082050 describes bacterial compositions and preparation methods thereof. In this document, after being concentrated and filtered, the bacterial cells are added with a protection solution containing gelatin, trehalose and a phosphate buffer. This document neither describes the use of pyrophosphate nor evaluates the advantages resulting from the use thereof. In a cryoprotection solution. Furthermore, this document does not describe a preparation method suitable to improve the viability and stability of bacterial cells.

Therefore, the need is felt to be able to have a method that is easy to carry out and to reproduce for preparing a biomass of freeze-dried, high-concentration, stable and viable bacterial cells capable of being transported, processed, marketed and stored in countries present in climatic zones IV.A and IV. B.

### SUMMARY OF THE INVENTION

Thus, the present invention falls in the context outlined above, setting out to provide (1) a biomass of freeze-dried, high-concentration and stable bacterial cells; (2) a method for preparing said biomass of freeze-dried, high-concentration and stable bacterial cells; and (3) a pharmaceutical composition, or a medical device composition, or a cosmetic use composition, or a food supplement composition or a food product composition or a food for special medical purposes (FSMP) composition (all of these compositions referred to, for the sake of brevity, as “compositions of the present invention”) comprising, said compositions, said biomass of freeze-dried, high-concentration and stable bacterial cells.

The freeze-dried bacterial cells, subject of the invention, are cells with a well-preserved cell wall (or ceil membrane wall) in a good physiological state and they therefore are integral and viable cells. The integrity of the cell wall (or of the call membrane wall) confers to the cells greater stability in terms of viability expressed in AFU and determined by means of a cytofluorometry method. The stability is greater than the stability determined on the same cells by means of plate count and expressed in CFU. A greater stability allows to have a biomass of bacterial cells with a prolonged shelf-life, while a greater cell viability allows to have a greater activity and effectiveness once used or administered to a subject being treated, Forming an object of the present invention is a biomass of freeze-dried, high-concentration and stable bacterial cells having the characteristics as defined in the attached claims.

Forming another object of the present invention is a method for preparing said biomass of freeze-dried, high-concentration and stable bacterial cells, having the characteristics as defined in the attached claims.

Still forming an object of the present invention is a pharmaceutical composition, or a medical device composition, or a cosmetic use composition, or a food supplement composition or a food product composition or a food for special medical purposes (FSMP) composition (all of these compositions referred to, for the sake of brevity, as “compositions of the present invention”) comprising, said compositions, said biomass of freeze-dried, high-concentration and stable bacterial cells, having the characteristics as defined in the attached claims

Forming an object of the present invention is a cryoprotection solution according to the attached claims.

Forming an object of the present invention is the use of the at least one pyrophosphate ion salt or pyrophosphoric acid and mixtures thereof, of the at least one polyhydroxy substance (b) and optionally, (c) L-cysteine for cryoprotecting a biomass of bacterial cells (bacterial biomass), according to the attached claims.

Preferred embodiments of the present invention are described in greater detail hereinafter without intending to limit the scope of protection of the present invention in any manner whatsoever.

### BRIEF DESCRIPTION OF THE DRAWINGS

The present invention will now be described with reference to the attached drawings, provided by way of non-limiting example, wherein:

FIG. 1 shows a first diagram according to Example 6, test A) discussed hereinafter;

FIG. 2 shows a second diagram according to Example 6, test B) discussed hereinafter;

FIG. 3 shows a third diagram according to Example 6, test C) discussed hereinafter;

FIG. 4 shows the decay rate (k) of Example 6, regarding ZONE IV.B., similar to the slope of the interpolation line

FIG. 5 shows the result of the pyrophosphate detection assay in the 6 samples, according to Example 7. In detail, FIG. 5A shows the first set of 6 samples, while FIG. 58 shows the second set of 6 samples. The first test tube both in (A) and (B) is the negative control (NEG=distiled water), the second test tube both in (A) and (B) is the positive control (POS=Potassium Pyrophosphate).

FIG. 6 shows the result of the sucrose detection assay in the 6 samples according to Example 7. In detail, FIGS. 6A and 6B show the first set, FIGS. 6C and 6D show the second set. The first test tube is the negative control (NEG=distilled water), the second test tube is the positive control (POS=Potassium Pyrophosphate).

FIG. 7 shows the ATR-FTIR spectra of sucrose and potassium pyrophosphate, according to Example 7.

FIG. 8 shows the ATR-FTIR spedra of the six samples (first set) according to Example 7. In the key: 1 corresponds to sample 1, 2 corresponds to sample 2, 3 corresponds to sample 3, 4 corresponds to sample 4, 5 corresponds to sample 5, and 6 corresponds to sample 6.

FIG. 9 shows the potentiometric titration of potassium pyrophosphate in the six liquid samples analysed, according to Example 7.

FIG. 10 shows the sucrose calibration curve y=205.5x−6.182 R²=0.999, according to Example 7.

FIG. 11 shows the HPLC chromatogram of a sucrose standard solution (5 mg/ml), according to Example 7.

FIG. 12 shows the HPLC chromatogram of a glucose standard solution (5 mg/ml) and the HPLC chromatogram of a fructose standard solution (5 mg/ml), according to Example 7.

FIG. 13 shows an example of HPLC chromatogram of sample 6, according to Example 7.

FIG. 14 shows the DCF calibration curve, according to Example 7.

### DETAILED DESCRIPTION OF THE INVENTION

After an intense and prolonged research and development activity, motivated and supported by several very promising experimental data, the Applicant has come to understand the importance of the wall of the bacterial cells (cell wall) present in a biomass (set of bacterial cells).

The experimental findings have confirmed that maintaining a good state of preservation and integrity of the cell wall during all the steps for preparing a biomass of freeze-dried bacterial cells allows to obtain stable, viable and high-concentration bacterial cells, by means of an optimised and reproducible process.

The above has been possible thanks to a specific method for preparing a biomass of freeze-dried bacterial cells, subject of the present invention. Furthermore, the above has also been possible thanks to a method, subject of the present invention, which provides for the combination of said preparation method with a method for evaluating the cell wall. The method for evaluating the cell wall, also subject of the present invention, allows to evaluate whether said cell wall is well preserved in a good physiological state. The preservation of a good physiological state and the integrity of the cell wall are important for the stability and viability of the cells.

The monitoring and evaluation of a good state of preservation and integrity of the cell wall, carried out in all steps of the method for preparing said biomass of bacterial cells, allows to optimise each of the individual steps of the preparation method with the aim of obtaining a biomass of freeze-dried, stable, viable and high-concentration bacterial cells, by means of a reproducible, reliable and optimised process.

Advantageously, the method comprising the preparation method of the present invention combined with the method for evaluating the maintenance of a good state of preservation and integrity of the cell wall has allowed to obtain a biomass of freeze-dried bacterial cells with an integral and well-preserved cell wall (membrane integrity) which confers a prolonged stability and an excellent viability to the freeze-dried bacterial cells.

In the context of the present invention, the term “integral” is used to indicate that the cell membrane or cell membrane wall does not have permeability elements or zones due to an increase in damage to the membrane.

The preparation method of the present invention improves the sealing of the cell membrane of the bacterium by reducing cell permeability.

The expression prolonged stability is used to indicate a shelf-life stability, determined by means of a cytofiluorometry method, which results to be greater than the stability of the same biomass of bacterial cells measured by means of the standard plate count method.

Furthermore, an integral and well-preserved cell wall (membrane integrity) confers a greater viability and effectiveness to the freeze-dried bacterial cells once said cells have been administered to a subject.

Thanks to the preparation method of the present invention, it is possible to prepare a biomass of bacterial cells in which the cells exhibit stability for a period of time comprised from 1 minute to 10 years, preferably comprised from 1 day to 5 years, more preferably comprised from 4 months or from 12 months to 48 months, even more preferably from 18 months to 32 months, further preferably from 24 months to 30 months, even under conditions of zone IV.A and zone IV.B.

The present invention regards a method for preparing a biomass of freeze-dried bacterial cells, comprising the following steps:

(i) fermenting a previously prepared biomass of bacterial cells (bacterial biomass) comprising at least one strain of bacterial cells to obtain a fermented biomass of bacterial cells (fermented biomass);

(ii) concentrating the fermented biomass obtained from step (i) up to obtaining a concentrated biomass of bacterial cells (concentrated biomass) having a bacterial cell concentration comprised from 1×10⁶ cells/ml of liquid biomass to 1×10¹² cells/ml of liquid biomass;

(iii) mixing the concentrated biomass obtained from step (ii) with a solution comprising, or alternatively, consisting of: (a) at least one phosphorous salt selected from among the group comprising or, alternatively, consisting of a phosphate ion salt or phosphoric acid, a phosphite ion salt or phosphorous acid, a monohydrogen phosphate ion salt, a dihydrogen phosphate ion salt, a pyrophosphate ion salt or pyrophosphoric acid, and the mixtures thereof, and (b) at least one polyhydroxy substance selected from among the group comprising or, alternatively, consisting of sucrose, fructose, lactose, lactitol, trehalose or mannitol, and the mixtures thereof, to obtain a cryoprotected biomass of bacterial cells (cryoprotected biomass);

(iv) freeze-drying the cryoprotected biomass obtained from step (iii) to obtain a biomass of freeze-dried bacterial cells (freeze-dried biomass). Advantageously, said (a) at least one phosphorus salt is a pyrophosphate ion salt or pyrophosphoric acid, for example sodium or potassium pyrophosphate.

In step (iii) the concentrated biomass of step (ii) may be mixed with a solution (cryoprotectant) comprising or, alternatively, consisting of (a) at least one phosphorus salt, (b) at least one polyhydroxy substance and (c) L-cysteine. Advantageously, said (a) at least one phosphorus salt is a pyrophosphate ion salt or pyrophosphoric acid, for example sodium or potassium pyrophosphate.

In step (iii) the cryoprotected biomass obtained from step (ii) can be mixed with a solution (cryoprotectant) comprising or, alternatively, consisting of (a) at least one pyrophosphate ion salt or pyrophosphoric acid and mixtures thereof, (b) at least one polyhydroxy substance, optionally (c) L-cysteine, and (d) at least one citric acid salt Where said citric acid salt can be a pharmacologically acceptable salt, for example it can be sodium citrate or potassium citrate or magnesium citrate or calcium citrate or mixtures thereof, preferably sodium and/or magnesium citrate and mixtures thereof.

Therefore, in a first embodiment, said solution (cryoprotectant) may comprise or, alternatively, consist of (a) at least one pyrophosphate ion salt or pyrophosphoric acid, such as for example sodium and/or potassium pyrophosphate, (b) at least one polyhydroxy substance, preferably sucrose, and/or trehalose and (d) at least one citric acid salt, preferably a sodium and/or potassium citrate. Whereas, in a second embodiment, said solution (cryoprotectant) may comprise or, alternatively, consist of (a) at least one pyrophosphate ion salt or pyrophosphoric acid, such as for example sodium and/or potassium pyrophosphate, (b) at least one polyhydroxy substance, preferably sucrose, and/or trehalose (c) L-cysteine, and (d) at least one citric acid salt, preferably a sodium and/or potassium citrate.

An example of cryoprotection solution (cryoprotectant) used in step (iii) may be a solution comprising (a) potassium and/or sodium pyrophosphate and mixtures thereof, (b) sucrose, optionally (c) cysteine and (d) sodium and/or magnesium citrate and mixtures thereof.

Another example of cryoprotection solution (cryoprotectant) according to the present invention may be a solution comprising (a) potassium and/or sodium pyrophosphate and mixtures thereof, (b) trehalose, optionally (c) L-cysteine and (d) sodium and/or magnesium citrate and mixtures thereof.

The cryoprotection solution according to the present invention may have a pH comprised from 8.5±0.1 to 9.8±0.1, preferably from 8.8±0.1 to 9.5±0.1, for example the pH of the cryoprotection solution may be 9.2±0.1.

For example, the cryoprotection solution comprising potassium pyrophosphate, sucrose and sodium citrate has a pH±9.2±0.1.

Besides steps (i), (ii), (iii) and (iv) the method of the present invention may also comprise one or more of the following preferred steps.

The fermented biomass obtained from step (i) may have a pH comprised from 3.0±0.1 to 6.0±0.1 preferably comprised from 5.0*0.1 to 6.0±0.1.

In a preferred embodiment, the method of the present invention may provide for a step (a) in which the pH of the fermented biomass obtained from step (i) is adjusted, if necessary, to a pH value comprised from 6.0±0.1 to 6.8±0.1, to obtain a fermented biomass at adjusted pH; preferably the pH value could be comprised from 6.2±0.1 to 6.5±0.1, for example the pH value could be 6.4±0.1. This step (i.a), if present, is carried out before step (ii). The measured pH values may have a measured comprised tolerance of ±0.1 or ±0.2.

According to an embodiment, the adjustment of the pH value on the fermented biomass is carried out by adding a weak base, preferably inorganic. Preferably, the weak base comprises or, alternatively, consists of ammonium hydrate (NH4OH; CAS No. 1336-21-6).

By way of example, an ammonium hydrate usable to adjust the pH value could be an aqueous solution with an ammonia titre of 31%-32%, and preferably with a specific weight of 0.887-0.890 g/cm3.

In a preferred embodiment, besides steps (i), (ii), the method of the present invention may further provide for a preferred step (ii.a) prior to step (iii). In the preferred step (ii.a) the concentrated biomass obtained from step (ii) is washed to obtain a washed biomass.

According to an embodiment, in step (ii.a) the concentrated biomass obtained from step (ii) is washed with a washing liquid, preferably water.

In a preferred embodiment, besides steps (i), (ii), (i.a), the method of the present invention may further provide for a preferred step (ii.b) prior to step (iii). In the preferred step (ii.b), the washed biomass obtained from step (ii.a) is re-concentrated to obtain a re-concentrated biomass.

In a re-concentrated biomass according to the present invention, the bacterial cells preferably have a concentration comprised from 1×10⁶ cells/ml to 1×10¹² cells/ml, preferably comprised from 1×10⁷ cell/ml to 1×10¹² cells/ml, even more preferably comprised from 1×10⁸ cells/ml to 1×10¹¹ cells/ml, more preferably still comprised from 1×10⁹ cells/ml to 1×10¹¹ cells/ml or comprised from 1×10¹⁰ cells/ml to 1×10¹¹ cells/ml, for each milliliter of re-concentrated liquid biomass.

The concentrated biomass obtained from step (ii), or the washed biomass obtained from step (ii.a), or the re-concentrated biomass obtained from step (ii.b) may have a pH comprised from 6.0*0.1 to 7.0±0.1, preferably comprised from 6.4±0.1 to 6.7±0.1.

In a preferred embodiment, the washed and re-concentrated biomass obtained from step (ii.a) and (ii.b) is mixed with a solution comprising or, alternatively, consisting of (a) at least one phosphorus salt selected from among the group comprising or, alternatively, consisting of a phosphate ion or phosphoric acid salt, a phosphite ion or phosphorous acid salt, a monohydrogen phosphate ion salt, a dihydrogen phosphate ion salt, a pyrophosphate ion salt or pyrophosphoric add, and mixtures thereof, and (b) at least one polyhydroxy substance selected from among the group comprising or, alternatively, consisting of sucrose, fructose, lactose, lactitol, trehalose, mannitol, and mixtures thereof, to obtain the cryoprotected biomass. Preferably, the solution comprises or, alternatively, consists of (a) at least one phosphorus salt, (b) at least one polyhydroxy substance, preferably also (c) L-cysteine.

In a preferred embodiment, the washed and re-concentrated biomass obtained from step (ii.a) and (ii.b) is mixed with a solution comprising or, alternatively, consisting of: (a) at least one pyrophosphate ion salt or pyrophosphoric acid, and mixtures thereof, and (b) at least one polyhydroxy substance selected from among the group comprising or, alternatively, consisting of sucrose, fructose, lactose, lactitol, trehalose, mannitol, and mixtures thereof, to obtain the cryoprotected biomass, and optionally (c) L-cysteine. Advantageously said (a) at least one pyrophosphate ion salt can be sodium or potassium pyrophosphate or mixtures thereof.

In a preferred embodiment, the washed and re-concentrated biomass obtained from step (ii.a) and (ii.b) is mixed with a solution comprising or, alternatively, consisting of: (a) at least one pyrophosphate ion salt or pyrophosphoric add, and mixtures thereof, and (b) at least one polyhydroxy substance selected from the group comprising or, alternatively, consisting of sucrose, fructose, lactose, lactitol, trehalose, mannitol, and mixtures thereof, to obtain the cryoprotected biomass, optionally (c) L-cysteine, and at least one citric acid salt, for example sodium citrate and/or potassium citrate. Advantageously said (a) at least one pyrophosphate ion salt can be sodium and/or potassium pyrophosphate and mixtures thereof.

In a preferred embodiment, besides steps (i), (ii), (ii.a) and (ii.b), the method of the present invention may further provide for a preferred step (ii.c) prior to step (iii). In the preferred step (ii.c) the pH of the re-concentrated biomass obtained from step (ii.b) is adjusted, if necessary, to a pH value comprised from 5±0.1 to 7±0.1, to obtain a biomass with adjusted pH; preferably the pH value could be comprised from 5.5±0.1 to 6.5±0.1, even more preferably the pH value could be of 6.2±0.1.

According to an embodiment, the pH value adjustment in step (ii.c) Is carried out by adding a weak, preferably inorganic, base. Preferably, the weak base comprises or, alternatively, consists of ammonium hydrate (NH₄OH; CAS No. 1336-21-6).

By way of example, an ammonium hydrate that can be used to adjust the pH value in step (ii.c) could be an aqueous solution with an ammonia titre of 31-32%, and preferably with a specific weight of 0.887-0.890 g/cm³.

Alternatively, should the pH be adjusted in step (i.a), the aforementioned step (ii.c) cannot be carried out.

In a preferred embodiment, the biomass washed, re-concentrated and with adjusted pH obtained from step (i), (i.a) (ii), (ii.a), (i.b) or from step (i), (ii.a), (ii.b) and (ii.c) is mixed with solution comprising or, alternatively, consisting of (a) at least one phosphorus salt selected from among the group comprising or, alternatively, consisting of a phosphate ion or phosphoric acid salt, a phosphite ion or phosphorous acid salt, a monohydrogen phosphate ion salt, a dihydrogen phosphate ion salt, a pyrophosphate ion salt or pyrophosphoric acid, and mixtures thereof, and (b) at least one polyhydroxy substance selected from among the group comprising or, alternatively, consisting of sucrose, fructose, lactose, lactitol, trehalose, mannitol, and mixtures thereof, to obtain the cryoprotected biomass. Preferably, the solution comprising or, alternatively, consisting of (a) at least one pyrophosphate ion salt or pyrophosphoric acid, and mixtures thereof, and, (b) at least one polyhydroxy substance, and preferably also (c) L-cysteine.

In step (iii)—subsequent to step (ii), or subsequent to step (ii.a) and (ii.b), or subsequent to step (ii.a), (ii.b) and (i.c)—the concentrated biomass obtained from step (ii), or the washed and re-concentrated biomass obtained from step (ii.a) and (ii b), or the biomass washed, re-concentrated and with adjusted pH obtained from step (ii.a), (ii.b) and (ii.c), is mixed with a solution comprising or, alternatively, consisting of: (a) at least one phosphorus salt selected from among the group comprising or, alternatively, consisting of a phosphate ion or phosphoric acid salt, a phosphite ion or phosphorous acid salt, a monohydrogen phosphate ion salt, a dihydrogen phosphate ion salt, a pyrophosphate ion salt or pyrophosphoric acid, and mixtures thereof, and (b) at least one polyhydroxy substance selected from among the group comprising or, alternatively, consisting of sucrose, fructose, lactose, lactitol, trehalose, mannitol, and mixtures thereof, to obtain the cryoprotected biomass. Preferably, the solution comprising or, alternatively, consisting of (a) at least one phosphorus salt. (b) at least one polyhydroxy substance may also further comprise (c) L-cysteine. Advantageously, said (a) at least one phosphorus salt is a pyrophosphate ion salt or pyrophosphoric acid and mixtures thereof.

Sodium or potassium pyrophosphate Na₄P₂O₇ or K₄O₇P₂ is a sodium or potassium salt of pyrophosphoric acid H₄P₂O₇. Sodium pyrophosphate is also called tetrasodium pyrophosphate to distinguish it from sodium acid pyrophosphate Na₂H₂P₂O₇. At room temperature, sodium or potassium pyrophosphate appears as a colourless, odourless, water-soluble solid. Together with the other sodium or potassium diphosphates it is encoded in the list of food additives as E450. Advantageously, in the cryoprotection solution according to the present invention, said pyrophosphate ion salt is sodium pyrophosphate and/or potassium pyrophosphate and mixtures thereof.

In the present invention, the at least one pyrophosphate ion salt or pyrophosphoric acid, and mixtures thereof, are advantageously used to cryoproprotect a biomass of bacterial cells. The use of the at least one pyrophosphate ion salt or pyrophosphoric add and mixtures thereof may occur in combination with at least one polyhydroxy substance. In an embodiment of the present invention, sodium and/or potassium pyrophosphate may be used in the solution (cryoprotectant) in combination with sucrose and/or trehalose.

The use of the at least one pyrophosphate ion salt or pyrophosphoric acid, and mixtures thereof in the cryoprotection solution allows to obtain a biomass of freeze-dried, high-concentration, stable and viable bacterial cells. Advantageously, said (a) at least one phosphorus salt is a pyrophosphate ion salt or pyrophosphoric acid and mixtures thereof, for example sodium pyrophosphate or potassium pyrophosphate and mixtures thereof.

The cryoprotected biomass obtained in step (iii) may have a pH comprised from 7±0.1 to 10±0.1, preferably comprised from 7±0.1 to 9±0.1, even more preferably comprised from 7.5±0.1 to 8.5±0.1.

Subsequently, the cryoprotected biomass obtained from step (iii) is freeze-dried according to step (iv) to obtain a biomass of freeze-dried, stable, viable and high-concentration bacterial cells, by means of an optimised and reproducible process.

It should be observed that in the present description the term “concentrated” in the expression “concentrated biomass” or in the expression “concentrated biomass of bacterial cells” is used to indicate a biomass obtained from step (ii) in which the bacterial cells are increased in number, per volume unit, with respect to those obtained at the end of the fermentation step (i).

In the concentrated biomass the bacterial cells have a concentration comprised from 1×10⁶ cells/ml to 1×10¹² cells/ml, preferably comprised from 1×10⁷ cells/ml to 1×10¹² cells/ml, even more preferably comprised from 1×10⁹ cells/ml to 1×10¹¹ cells/MA, more preferably still comprised from 1×10⁹ cells/ml to 1×10¹¹ cells/ml or comprised from 1×10¹⁰ cells/ml to 1×10¹¹ cells/ml, for each milllitre of concentrated liquid biomass.

When the biomass of bacterial cells is produced in solid phase following a drying process (for example flakes, granules or powder) or a freeze-drying process (for example freeze-dried powder), the term ‘concentrated’ as described in this description, will be used to indicate a bacterial cell concentration comprised from 1×10⁶ cells/g to 1×10³ cells/g, preferably a concentration comprised from 1×10⁷ cells/g to 1×10¹² cells/g, even more preferably a concentration comprised from 1×10⁸ cells/g to 1×10¹² cells/g, more preferably still a concentration comprised from 1×10⁹ cells/g to 1×10¹² cells/g, for each gram of dried biomass, or of freeze-dried biomass obtained from step (iv).

As regards the activity value of activity water Aw which allows, the lower the value, to reduce/inhibit the metabolic activity of the bacterial cells, it is important that the value of Aw, present in the freeze-dried biomass obtained from step (iv), be comprised from 0.01 to 0.3; preferably from 0.05 to 0.2; even more preferably from 0.1 to 0.15. The measurement and determination of the activity value of activity water Aw can be carried out using the “AQUALAB 4TE” instrument model, produced by the US company METER Group, Inc.

Dew-point on a cooled mirror is the technique used by the “AQUALAB 4TE” instrument. According to such technique, a sample to be analysed is introduced into a chamber of the instrument, subsequently hermetically sealed, and the humidity conditions of the chamber are progressively brought into equilibrium using the ‘activity water’ of said sample (defined as water not bound by cell bonds to the biomass bacterial cells). The instrument further comprises at least one thermoregulated mirror, inserted in the hermetically sealed chamber, and one or more detection sensors functionally connected to the thermoregulated mirror. During the analysis, upon reaching the equilibrium conditions between the chamber and the sample, a surface of the thermoregulated mirror is progressively brought to a temperature equal to or lower than the dew-point temperature of the humidity at the internal pressure of the chamber. The humidity of the chamber is then deposited on this surface of the thermoregulated mirror in the form of condensation. The detection sensor then detects a first condensation on the surface of the mirror, so that the instrument can detect the water activity Aw (which corresponds to the activity water of the sample) and the temperature of the surface of the mirror at which the first condensation occurred.

The method for preparing the freeze-dried biomass according to the present invention comprises the step (i) in which a biomass of bacterial cells (bacterial biomass) prepared previously and comprising at least one strain of bacterial cells is fermented to obtain a biomass of fermented bacterial cells (fermented biomass).

The bacterial biomass intended for step (i) comprises at least one strain of bacterial cells selected from among the group comprising or, alternatively, consisting of strains of bacterial cells belonging to the families' Firmicutes, Acibactera, Bacteroidetes, Proteobacteria, and mixtures thereof. Said at least one strain of bacterial cells is selected from among the group comprising or, alternatively, consisting of strains of bacterial cells belonging to the genera: Lactobacilus, Bifidobacterium, Streptococcus, Lactococcus, Akkemansia, Intesfinimonas, Eubacterium, Faecalibacterium, Neisseria, Roseburia, Cutibacterium and mixtures thereof. Said at least one strain of bacterial cells is selected from among the group comprising or, alternatively, consisting of strains of bacterial cells belonging to the species: Lactobacillus acidophilus, Lactobacillus buchneri, Lactobacillus fermentum, Lactobacillus salivarius subsp. salivarius, Lactobacillus crispatus, Lactobacillus paracasel subsp. paracasei, Lactobacillus gasseri, Lactobacillus plantarum, Lactobacillus delbrueckii subsp. bulgaricus, Lactobacillus delbrueckii subsp. delbrueckii, Lactobacillus rhamnosus, Lactobecillus pentosus, Lactobacdius fermentum, Lactobacilus brevis, Lactobacillus casei, Lactobacillus reuteri, Lactobacillus johnsonii. Bifidobacterium adolescentis, Bifidobacterium animals subsp. lactis, Bifobacterium breve, Bifobacterium catenulatum, Bifobacterium pseudocatenulaum, Bindobacterium bifidum, Bifdobacterium lactis, Bifidobacterium infantis, Bifldobactrium longum, Akkermansia munichipila, Intestinimonas butynciproducens, Eubacterium hallii, Faecalibacterium prausnitzii, Neisseda lactamica, Roseburia hominis, Cutibacterum acnes, and mixtures thereof.

In an embodiment, a bacterial biomass intended for step (i) of the strain of bacteria of interest is inoculated into a liquid fermentation substrate (or fermentation broth) comprising: i) a carbon source, preferably dextrose at a concentration comprised from 20 g/l to 80 g/l, ii) a nitrogen source, preferably comprising a combination of a peptone of plant origin (for example, potato, rice or pea as a function of the strain of fermented bacteria), and iii) a yeast extract at a concentration comprised from 5 g/l to 50 g/l According to an embodiment, the liquid fermentation substrate can be added, as a function of the biomass of bacteria of interest, with phosphate salts or potassium, magnesium or manganese sulphate at the concentration of each of such salts preferably comprised from 10 g/l to 0.01 g/l of said substrate or fermentation broth.

The bacterial biomass of interest is inoculated into the liquid fermentation substrate described above amounting to 1-10%, preferably 2-4%, by volume with respect to the volume of the liquid fermentation substrate.

The bacterial biomass thus inoculated is incubated at a temperature comprised from 30° C. to 40° C., preferably from 34° C. to 37° C., for a period of time comprised from 1 hour to 48 hours, preferably from 5 hours to 30 hours, as a function of the inoculation and acidification of the liquid fermentation substrate.

At the end of the fermentation step (i) there is obtained a bacterial biomass which is subjected to a concentration according to step (ii).

Step (ii), in which the bacterial biomass of step (i) is concentrated, is implemented by means of a separation step, in which a liquid fraction is separated from a solid or cellular fraction consisting precisely of the bacterial cells grown in the liquid fermentation substrate of step (i). In an embodiment, said separation step can be carried out by means of centrifugation.

The separation step allows to separate from the bacterial biomass, which is in the physical state of a solution, the liquid fraction contained therein so that the biomass increasingly focuses on the other components such as, for example, bacterial cells.

The concentration is achieved by passing from a bacterial biomass which contains, in step (i), said at least one bacterial strain at a concentration comprised from 1×10⁶ cells/ml to 1=10¹¹ cells/ml of substrate or fermentation broth, to a concentrated biomass containing, after step (i), said at least one bacterial strain at a concentration comprised from 1×10⁶ cells/ml to 1×10¹ cells/ml.

The preferred step (ii.a), additional to steps (i), (i) and preceding step (iii), provides for that the concentrated biomass obtained from step (ii) is washed with the washing liquid, preferably water, to obtain the washed biomass.

In step (iii) the concentrated biomass obtained from step (ii), or the biomass with adjusted pH obtained from step (ii.b), is mixed with the solution comprising or, alternatively, consisting of (a) and (b), and optionally (c).

Such a solution (or cryoprotection solution) is capable of conferring to the concentrated biomass, or to the washed biomass, or to the biomass with adjusted pH, or to the re-concentrated biomass, a cryoprotection in the sense that the bacterial biomass is cryoprotected. This means that the cells of the bacterial strain used, contained in said bacterial biomass, are cryoprotected. Cell cryoprotection means that the biological tissues (for example the cell membrane) of the cells of the bacterial strain are protected from possible damage resulting from freezing in the step (iv) for freeze-drying the cryoprotected biomass. By way of example, damage to the cells could comprise a laceration or a lesion of the cell membrane, accompanied by a possible increase in permeability through the membrane.

According to an embodiment, said solution of step (iii) is an aqueous solution, for example distilled or bidistilled water at room temperature of 20° C.-25° C.

According to an embodiment, the phosphorus salt (a) is a pyrophosphate salt.

According to another embodiment, at least one phosphorus salt (a) is selected from among the compounds of potassium phosphate (K₃PO₄), potassium monohydrogen phosphate (K₂HPO₄), potassium dihydrogen phosphate (KH₂PO₄) and/or potassium pyrophosphate (K₄P₂O₇) By way of example, a potassium monohydrogen phosphate that can be used in this invention is in the form of white crystals and it has a titre comprised from 90% to 100% by weight, preferably comprised from 95% to 99% by weight, even more preferably comprised from 97% to 99% by weight.

By way of further example, a potassium pyrophosphate (CAS No. 7320-34-5) that can be used in this invention is in the form of particles, powder or granules and has a titre comprised from 90% to 100% by weight, preferably comprised from 95% to 99% by weight, even more preferably comprised from 96% to 98% by weight.

According to an embodiment, the phosphate ions, the monohydrogen phosphate ions, the dihydrogen phosphate ions and/or the pyrophosphate ions could be present in the solution (considering such solution before the mixing thereof with the bacterial biomass in step (iii)) at an amount comprised from 6 to 27% W/V, where % W/V is used to indicate a percentage by weight (i.e. grams) of the aforementioned compounds with respect to the total volume of the solution.

According to an embodiment, the concentration of phosphorus salt or salts (a) in the solution used in step (iii) could be comprised from 6 to 20% W/V, preferably comprised from 6 to 15% W/V, even more preferably comprised from 10 to 14% W/V.

The polyhydroxy substance (b) is selected from among the group comprising or, alternatively, consisting of sucrose, fructose, lactose, lactitol, trehalose or mannitol, and mixtures thereof.

According to an embodiment, sucrose could be used as a polyhydroxy substance. According to an embodiment, sucrose could have a concentration comprised from 25% W/V to 45% W/V, where % W/V is used to indicate a percentage by weight (i.e. grams) of sucrose with respect to the total volume of the cryoprotection solution (considering such solution before mixing it with the bacterial biomass. In step (Ili)).

By way of example, a sucrose usable as a polyhydroxy substance (b) could be in the form of white and water-soluble crystals. Preferably, a percentage by weight comprised from 85% to 100%, preferably comprised from 90% to 95%, of the sucrose crystals has a particle size distribution comprised from 0.05 to 0.50 millimetres, preferably comprised from 0.1 to 0.35 millimetres.

Thus, according to an embodiment, the solution used. In step (iii) could comprise a solvent (preferably water), pyrophosphate ions and phosphate ions, monohydrogen phosphate ions, and/or dihydrogen phosphate ions, preferably L-cysteine, and sucrose.

According to an embodiment, L-cysteine in the solution used in step (iii) could be present at an amount comprised from 0.5 grams of L-cysteine to 5 grams of L-cysteine per each litre of solution, preferably comprised from 1 gram to 4 grams of L-cysteine per each litre of solution, even more preferably comprised from 2 grams to 3 grams of L-cysteine per each litre of solution.

Specifically, L-cysteine (CAS No. 52-90-4) serves as an oxygen sequestrant and therefore, when added to the solution, it limits or prevents the formation of reactive oxygen species (ROS). Furthermore, L-cysteine is characterised by allow molecular weight, and therefore it can easily penetrate the cell membranes of the bacterial cells, thus increasing protection from damage resulting from oxygen free radicals, and improving the integrity of the membrane structure.

By way of example, an L-cysteine that can be used within the scope of the present invention could be monohydrate.

According to an embodiment, an L-cysteine that can be used in this invention is in the form of crystalline powder and it has a titre comprised from 90% to 100% by weight, preferably comprised from 95% to 100%.

According to an embodiment, the solution used in step (Iii) could comprise pyrophosphate ions and sucrose at a pyrophosphate-sucrose ion molar ratio comprised from about 1:1.5 to about 1:6, preferably 1:3.

According to a further embodiment, the solution used in step (iii) could comprise phosphate ion, monohydrogen phosphate ion, dihydrogen phosphate ion and sucrose at a phosphate, monohydrogen phosphate, dihydrogen phosphate:sucrose ion molar ratio comprised from about 1:0.75 to about 1:3, preferably 1:1.5.

Thus, according to such embodiment, the solution of step (iii) could comprise a solvent (preferably water), pyrophosphate ions and/or phosphate ions, monohydrogen phosphate ions, dihydrogen phosphate ions, sucrose, and preferably L-cysteine.

In step (iv) the cryoprotected biomass obtained from step (iii) is freeze-dried to obtain a freeze-dried biomass.

Unless otherwise indicated, the expression “to freeze-dry” or “freeze-drying” will be used to indicate a controlled dehydration of the pre-frozen cryoprotected biomass, and it will be used to indicate the entire freeze-drying process (freezing, primary drying and secondary drying).

Example 4 describes a freeze-drying process according to a possible embodiment of this invention.

According to an embodiment, the freeze-drying of step (iv) comprises, after step (iii), the following steps: (iv.a) freezing the cryoprotected biomass obtained from step (ii) to obtain a frozen biomass; (iv.b) subliming the ice (or drying) of the frozen biomass obtained from step (iv.a) to obtain the freeze-dried biomass.

Preferably, the sublimation of step (iv.b) comprises a primary drying step (iv.b.1) of the frozen biomass obtained from step (iv.a), and a subsequent secondary drying (or desorption) (iv.b.2), on the biomass obtained from step (iv.1), to obtain the freeze-dried biomass.

In the primary drying step (iv.b.1), the frozen biomass obtained from step (iv.a) is initially subjected to a reduced pressure, so as to sublimate a part of the frozen solution, to obtain a biomass at reduced pressure and subsequently, in the secondary drying step (iv.b.2), the biomass at reduced pressure is heated to obtain the freeze-dried biomass Preferably, the secondary drying step (iv.b.2) starts when all the ice is sublimated from the biomass at reduced pressure in the previous primary drying step (iv.b.1).

In the secondary drying step (iv.b.2) the solution adsorbed on the biomass at reduced pressure obtained from the primary drying step (iv.b.1) is desorbed, by increasing the biomass temperature at reduced pressure.

According to an embodiment, the secondary drying step (iv.b.2) ends when the humidity of the biomass is comprised from 0.5% to 2.5% by weight, preferably comprised from 0.75% to 2.0% by weight, more preferably comprised from 0.9% to 1.5% by weight, even more preferably compnsed from 0.95% to 1.1% by weight of the biomass.

Besides steps (i), (ii), (iii) and (iv), according to an embodiment the method may comprise a step (v) subsequent to step (iv).

In the preferred step (v) the freeze-dried biomass obtained from step (iv) is crushed to obtain a crushed biomass.

As a matter of fact, the freeze-dried biomass obtained from step (iv) is a compact mass (cake), which mass must be crushed, ground, or broken up, to obtain the crushed biomass Preferably, the crushing of step (v) is carried out by means of a mesh or a sieve.

More precisely, in the preferred step (v), the compact mass or cake obtained from step (iv) is forced through the aforementioned mesh or through the aforementioned sieve in order to crush, grind, or break up the compact mass.

A crushed biomass, obtained at the end of step (v), is in the form of powder or granule, and it is easier to manage and handle with respect to the freeze-dried biomass of step (iv). For example, such improved handling may be useful in subsequent weighing and/or packaging operations.

Besides steps (i), (ii), (iii) and (iv), according to an embodiment the method may comprise a step (vi) subsequent to step (v).

In the preferred step (vi), the crushed biomass obtained from step (v) is packaged in a sterile container, preferably in the absence of moisture, to obtain a packaged biomass.

In an embodiment, the packaged biomass obtained from step (vi) is packaged in the sterile container so that the amount of head space in the sterile container (specifically, the amount of air between the packaged biomass and the top of the container) is very small. Preferably, the amount of head space is negligible (i.e., almost zero).

According to an embodiment, the packaged biomass has a bacterial cell concentration comprised from 1×10⁸ cells/g to 1×10¹¹ cells/g, preferably a concentration comprised from 1×10⁹ cells/g to 1×10¹⁰ cells/g, per each gram of packaged biomass obtained at the end of step (vi).

Besides steps (i), (ii). (ii) and (iv), according to an embodiment the method may comprise a step (vii) subsequent to step (vi).

In the preferred step (vii), the packaged biomass obtained from step (vi) is reconstituted with water after a predetermined time to obtain a reconstituted biomass.

With respect to the predefined time of step (vii), such time is preferably comprised from 1 minute to 10 years, preferably comprised from 1 day to 5 years, more preferably comprised from 4 months or from 12 months to 48 months, even more preferably from 18 months to 32 months, further preferably from 24 months to 30 months, even under conditions of Zone IV.A and Zone IV.B.

According to an embodiment, the reconstitution of step (vii) provides for a re-addition of a volume of water to the packaged biomass obtained from step (vi), typically, but not necessarily, equivalent to the volume reduced during freeze-drying of step (iv).

According to different embodiments, the water used in step (vii) is selected from among the group comprising or, alternatively, consisting of pure water, saline solution, or buffer solution.

According to an embodiment, the packaged freeze-dried biomass obtained from step (vi) could be reconstituted (hydrated) in step (vii) as an aqueous solution, preferably by means of an isotonic aqueous solution, even more preferably at a substantially neutral pH value or in any case comprised from 6.0 to 7.0. Such pH value comprised from 6.0 to 7.0 is particularly preferred for a packaged biomass obtained from step (vi) in which the bacterial cells are naked cells, i.e. devoid of an outer lining.

According to an embodiment, the packaged freeze-dried biomass obtained from step (vi) could be reconstituted (hydrated) in step (vii) as an aqueous solution of a borate buffer solution at pH 8.4. Such pH value 8.4 is particularly preferred for a packaged biomass obtained from step (vi) in which the bacterial cells are micro-encapsulated cells, preferably in a lipid matrix or in a glycoprotein matrix.

According to an embodiment, in the reconstitution of step (vii), the packaged biomass obtained from step (vi) is diluted up to obtaining a bacterial cell concentration in the reconstituted biomass comprised from 10⁵ to 10⁷ cells/ml, preferably about 10⁶ cells/ml.

In this regard, the bacterial cell concentration. In the reconstituted biomass comprised from 10⁵ to 10⁷ cells/ml, preferably about 10⁶ cells/ml, is preferably obtained by subsequent dilutions with water.

Besides steps (i), (ii), (iii) and (iv), according to an embodiment the method may comprise the preferred steps of

(viii) placing at contact the fermented biomass obtained from step (i), the concentrated biomass obtained from step (i), the cryoprotected biomass obtained from step (iii), and the freeze-dried biomass obtained from step (iv) with two different fluorescent dyes, so as to obtain a fluorescent fermented biomass, a fluorescent concentrated biomass, a fluorescent cryoprotected biomass and a fluorescent freeze-dried biomass (indicated in its entirety with the expression “fluorescent biomasses”);

(ix) subsequently to step (viii), by means of flow cytofluorometry, detecting an amount of bacterial cells with integral cell membranes (and thus viable) in the fluorescent fermented biomass, in the fluorescent concentrated biomass, in the fluorescent cryoprotected biomass and in the fluorescent freeze-dried biomass.

Therefore, the method according to this embodiment, in which a cytofluorometry detection of fluorescent biomasses is carried out in the different steps for preparing the freeze-dried biomass, allows to monitor (and therefore intervene/adjust in an improved manner) the parameters that govern step (i), step (ii), step (iii) and step (iv).

According to an embodiment, in the detection step of step (ix) and according to the method set forth in the ISO 19344:2015(E) standard, a first dye permeable through the cell membranes (preferably: thiazole orange or, alternatively, SYTO® 24—a fluorescent dye in the green spectrum) is capable of penetrating into all bacterial cells, providing the total fluorescent units or cells (TFU) of the fluorescent biomasses. A second dye (preferably: propidium iodide) is capable of penetrating only into the bacterial cells with a damaged cell membrane, providing the non-active or non-viable fluorescent units or ceNs (nAFU) of the fluorescent biomasses.

According to a particularly preferred embodiment, the amount of viable bacterial cells, with whole cell membranes, can be expressed as active fluorescent units or cells (AFU), i.e. units that are only positive to the first dye in fluorescence analysis (preferably: thiazole orange or, alternatively. SYTO® 24), for which the following correlation applies:

TFU=AFU+nAFU

where:
TFUs are the total fluorescent bacterial units or cells;
nAFUs are the non-active fluorescent bacterial units or cells units, with a non-Integral or damaged cell membrane (i.e. the units which are positive to the second dye, preferably propidium iodide).

According to an embodiment, the flow cytofluorometry of step (ix) is configured and/or calibrated to perform volumetric determination of the fluorescent biomasses analysed, and to directly calculate the cell concentration (AFU and TFU).

According to another embodiment, in order to obtain the values of AFU and TFU in the fluorescent biomasses, the flow cytofluorometry of step (ax) uses at least one internal fluorescent standard added to the fluorescent biomasses.

According to an embodiment, the internal fluorescent standard is in the form of a fluorescent ball or bead and it is added to each fluorescent biomass to be analysed in known concentration. The value of AFU and TFU in the fluorescent biomass analysed can then be calculated by proportion to the known amounts.

According to a further embodiment, the solution or cryoprotection solution is free of polymers having a molecular weight of from about 5,000 u to about 80,000 u, and/or the phosphate ions possibly present in the solution mixed with the concentrated biomass of step (ii) are not part of a buffer solution.

The aforementioned objectives are achieved by means of a freeze-dried biomass obtained by means of the method according to any of the embodiments discussed above.

According to an embodiment, such freeze-dried biomass is in solid form, preferably in the form of granule or powder.

The aforementioned objectives are lastly achieved by means of a pharmaceutical composition, or a medical device composition, or a cosmetic use composition, or a food supplement composition or a food product composition or a food for special medical purposes (FSMP) composition comprising, said compositions, the freeze-dried biomass according to any of the embodiments discussed above.

According to an embodiment, the compositions of the present invention comprise or, alternatively, consist of a Live Biotherapeutic Product (LBP), such expression being used to indicate a biological composition containing bacterial cells (particularly viable) and at least one drug or active ingredient, applicable for the treatment, for the prevention or for the cure of a disorder, of a disease or of a condition, and which does not comprise or consist of an immunogen-specific vaccine.

Hereinafter, the present invention will be illustrated based on some examples, solely provided by way of non-limiting example.

### EXAMPLES

### Example 1: Preparation of a Solution or Cryoprotection Solution that can be Used in Step (iii)

The following raw materials are poured into a container of suitable volume, measuring one litre, at the indicated ratios:
sucrose: 400 g/l;
sodium citrate: 50 g/l;
potassium monohydrogen phosphate: 135 g/l
L-cysteine: 2.5 g/l.

A sucrose that can be used in this Invention is SUCROSE RFF EP, cod. 649400, produced by Suedzucker AG, marketed by Giusto Faravelli S.p.A. (www.faravelli.it).

A sodium citrate that can be used in this invention is in the form of water-soluble crystals. Preferably, a percentage by weight comprised from 85% to 100%, preferably comprised from 90% to 95%, of the sodium citrate crystals has a particle size distribution comprised from 149 micrometres to 595 micrometres.

By way of example, a sodium citrate that can be used in this invention is SODIUM CITRATE TRIB.2H2O FINE CRYST. E331-BP-USPINF-EP, code 674500, produced by S.A. Citrique Beige N.V., marketed by Giusto Faravelli S.p.A. (www.faravelli.it).

A potassium monohydrogen phosphate that can be used in this invention is POTASSIUM PHOSPHATE BIB.ANHYDROUS E340, cod. 593500, marketed by Giusto Faravelli S.p.A (www.faravelli.it).

A L-cysteine that can be used in this invention is L-CYSTEINE HCL MONOHYDRATE produced by fermentation, code 285400, marketed by Giusto Faravelli S.p.A (www.faravelli.it).

The solution thus obtained is stirred until the raw materials are completely dissolved.

In a subsequent step, such solution is sterilised, in particular by thermal means. More precisely, such solution is heated (pasteurised) to a temperature of about 90° C., and maintained at such temperature for about 30-35 minutes.

Thereafter, the solution is cooled up to a temperature of about 6° C.-8° C. and it is thus ready for use.

During cooling, the solution can be insufflated with gaseous nitrogen to remove the dissolved oxygen and thus improve the compatibility of the cryoprotection solution with strictly anaerobic micro-organisms.

### Example 2: Preparation of Another Cryoprotection Solution that can be Used in Step (iii)

One proceeds as in Example 1, using about 128 g/l of alkaline pyrophosphate, preferably potassium pyrophosphate (CAS No 7320-34-5), instead of potassium monohydrogen phosphate, the solvent and the other raw materials remaining intact even in terms of ratios.

A potassium pyrophosphate that can be used in this invention is “Potassium pyrophosphate 97%”, product number 322431, marketed by Sigma-Aldrich (Saint Louis, Mo. 63103, United States; sigma-aldrich.com).

### Example 2A: Preparation of Another Cryoprotection Solution that can be Used in Step (iii)

One proceeds as in Example 1, using about 128 g/l of alkaline pyrophosphate, preferably potassium pyrophosphate (CAS No. 7320-34-5), instead of potassium monohydrogen phosphate, the solvent and the other raw materials remaining intact even in terms of ratios. In this example L-cysteine was not used.

A potassium pyrophosphate that can be used in this invention is “Potassium pyrophosphate 97%”, product number 322431, marketed by Sigma-Aldrich (Saint Louis, Mo. 63103, United States; sigma-aldrich.com).

### Example 2B: Preparation of Another Cryoprotection Solution that can be Used

In step (iii) comprising:
trehalose: 350 g/l;
sodium citrate: 50 g/l;
potassium pyrophosphate 128 g/l.

### Example 2C: Preparation of Another Cryoprotection Solution that can be Used in Step (iii) Comprising

trehalose: 350 g/l;
sodium citrate. 50 g/l;
potassium pyrophosphate 128 g/l;
L-cysteine: 2.5 g/l.

### Example 3: Preparing a Biomass Fermented According to Step (i) and a Biomass Concentrated According to Step (ii)

Starting from a culture of viable bacterial cells (a case of bacterial biomass) containing a strain of Lactobacillus rhamnosus GG (ATCC 53103), fermentation is carried out in a suitable fermentation substrate (or broth) for about 16-18 hours.

By way of example, an active culture of the aforementioned strain is inoculated amounting to 2-4% V/V (percentage by volume of the culture with respect to the volume of the substrate), preferably 3%; in the fermentation substrate consisting of dextrose, plant peptone and yeast extract in the amounts indicated above, plus manganese salts and surfactant. The culture is incubated at 31° C.-33° C. for about 16 hours, keeping the pH constant between 5.45 and 6.0 preferably between 5.80 and 5.90.

At the end of the fermentation step (i), a step (i.a) in which the pH of the fermented biomass is adjusted to 6.2±0.1 is carried out with a weak base preferably inorganic (preferably NH₄OH).

Subsequently, a first concentration (step (ii)) of the fermented biomass is carried out, specifically by centrifuging the aforementioned fermentation broth, and separating the aqueous phase from the solid or cellular phase.

The micro-organisms contained in the solid phase can then be washed (step (ii.a)), using sterile water (preferably bi-distilled) in a 4:1 ratio with respect to the weight of the bacterial biomass.

By means of a second centrifugation of the bacterial biomass mixed with sterile water in the aforementioned ratio, the washed biomass is then concentrated again (step ii.b)), with an overall volume concentration factor (VCF) comprised from about 10 to 30 times, preferably of about 20 times. This means that the final volume is reduced by about 10-30 times, preferably about 20 times, with respect to the initial volume, considering the same bacterial cells contained therein.

A washed and re-concentrated biomass is then obtained.

Alternatively, should step (i.a) not be carried out, the pH value of the washed and re-concentrated biomass can be adjusted (step (ii.c)), by adding a weak base, preferably inorganic base (preferably NH₄OH), to a pH of about 6.2*0.1 in order to obtain a biomass with adjusted pH.

An ammonium hydroxide that can be used in this invention is AMMONIUM HYDRATE, marketed by Flli Bonafede S.a.s. (21013 Gallarate (VA), Italy).

### Example 4: Mixing Step—Step (iii), and Freeze-Drying Step—Step (iv)

The cryoprotection solution of Example 1 (or example 2) is then added to biomass with the adjusted pH of Example 3 thus obtaining the cryoprotected biomass (CB) as a product of step (iii).

The ratio between the weight of the biomass at pH 6.2*01 and the volume of the cryoprotection solution could be comprised from about 80:20 to 75:25, after which freeze-drying is carried out. This means mixing the cryoprotection solution of Example 1 (or of Example 2) amounting to 20% calculated on the volume of the overall final mixture, or preferably amounting to 25% still calculated on the volume of the overall final mixture called cryoprotected biomass (CB).

CB is then loaded, i.e. placed in a freeze-dryer and subjected to a freeze-drying process called “freeze-drying” (lyophilisation).

To this end, the temperature of the cryoprotected biomass is lowered progressively in order to facilitate a complete freezing of the cryoprotected biomass (step (iv.a)) to obtain a frozen biomass. Specifically, the product is cooled up to a temperature comprised from −40° C. to −45° C. reached progressively (about 1° C.4 min), over a period of time of about 2 hours, and it is then kept frozen at the aforementioned temperature for about 2-4 hours.

Following such complete freezing (iv.a), the pressure of the chamber is reduced to a value of about 5.00E−02-5.00E−03 mbar, preferably 1.00E−03 mbar (step (iv.b.1)).

By maintaining this pressure value, the temperature is then raised again (step (iv.b.2)) in order to cause a sublimation of the cryoprotection solution. The phenomenon of sublimation is basically due to the fact that, below the triple point of the state diagram of such mixture, the solution solidified by freezing can modify the aggregation state thereof only in the gas phase, without liquefying.

For example, the heating ramp applicable to the product provides for that it be progressively brought from −45° C. to a temperature comprised from −20° C. to −10° C. in about 8 to 10 hours, for example by increasing the temperature with a step of 5° C., then maintained at a temperature of about −10° C. for another 4-8 hours. There follow at least two further heating steps first at 0° C. and maintaining this temperature for about 4 hours, and then up to about 15° C. maintained for approximately another 4 hours. The product is then brought to the final temperature of about 25°−30° C. at a rate of 0.5° C./min, and maintained at said final temperature for about 8 hours-12 hours.

The freeze-drying process (step (iv)) generally lasts 2-3 days, depending on the strain involved. In the present case of Lactobacillus rhamnosus GG (ATCC 53103), the freeze-drying process lasted about 60-72 hours.

The freeze-dried biomass thus obtained can be preserved, preferably after appropriate crushing/grinding (step (v)) of the product (cake) obtained at the end of the freeze-drying process, said preservation can optionally be carried out following a packaging step (step (vi)) in units or doses as indicated below in Example 6.

### Example 5: Analysis of the Freeze-Dried Biomass

The samples obtained were then analysed following the procedure illustrated in Example 4.

In the following Table 1, in the columns from left to right, there are reported the types of analyses carried out, the requirements or values obtained in the analyses, and the methods applied to test the quantities or values:

TABLE 1

| Analysis | Requirement | Test method |
|---|---|---|
| Physical examination | | |
| Appearance | Homogeneous white | Met. Int.*⁵ 203 (visual evaluation of the colour of the |
| | to off-white powder | powder by comparison with an internal reference |
| | | consisting of three shades of white. The product is |
| | | correct if its colour is within the reference) |
| Water activity (Aw) | ≤0.200 | Met. Int.*⁵ 201 (analysis carried out with a suitable |
| | | Aqualab instrument which allows to determine the water |
| | | activity of the sample based on the dew-point) |
| Assay/Potency | | |
| Viable cells | ≥5 × 10⁹ CFU*³/ | Met. Int.*⁵ 014 (direct plate count method by |
| Lactobacillus | dose (2 g) | reconstituting the sample in phosphate buffer, its |
| rhamnosus GG | | decimal dilutions in saline solution and inoculation of |
| (Plate Count, PC) | | appropriate dilutions in LAPTg agar medium) |
| Viable cells | ≥5 × 10⁹ AFU*⁴/ | ISO 19344:2015 |
| Lactobacillus | dose (2 g) | |
| rhamnosus GG | | |
| (FCM*¹) | | |
| Purity | | |
| Total aerobic | ≤1 × 10³ CFU*³/g | Met. Int*⁵ 004 according to Ph. Eur.*² 2.6.12 |
| microbial count | (compliant according | |
| (TAMC) | to Ph. Eur.*² 5.1.4 for | |
| | non-aqueous | |
| | preparations for oral | |
| | use) | |
| Total yeast and mould | ≤1 × 10² CFU*³/g | Ph. Eur.*² 2.6.12 |
| count | (compliant according | |
| (TYMC) | to Ph. Eur.*² 5.1.4 for | |
| | non-aqueous | |
| | preparations for oral | |
| | use) | |
| Gram-negative bile- | <100 CFU*³/g | Ph. Eur.*² 2.6.13, (4-1.) |
| tolerant bacteria | | |
| Staphylococcus | absent/g | Ph. Eur.*² 2.6.13, (4-5.) |
| aureus | | |
| Escherichia coli | absent/g | Ph. Eur.*² 2.6.13, (4-2.) |
| | (compliant according | |
| | to Ph. Eur.*² 5.1.4 for | |
| | non-aqueous | |
| | preparations for oral | |
| | use) | |
| Salmonella spp. | absent/10 g | Ph. Eur.*² 2.6.13, (4-3). |
| | (compliant) | |

*¹FCM = flow cytofluorometry;
*²Ph. Eur. = European Pharmacopoeia;
*³CFU = colony forming units;
*⁴AFU = active fluorescent units;
*⁵Met. Int. = internal method.
It should be observed that all the above standards are in the version valid at the priority date of this patent application.

### Example 6: Shelf Life Analysis

The product of Example 5 was preserved in a primary paper and aluminium packaging with the following stratifications, from the outside of the packet to the inside: a layer of paper (40 g/m²), two layers of aluminium each with a thickness of 9 μm, and a layer of polyethylene (thickness: 35 μm) directly in contact with the composition.

A cardboard box was used as a secondary packaging housing the primary packaging.

The parameters reported in the tables A), B), C) below were used in the tests marked with A), B), C) for the indicated periods of time (expressed in months), simulating the conditions of the following climatic zones (according to the WHO Technical report series No 953, 2009 guidelines, Annex 2, Appendix 1, Table 1, page 117):

A) ZONE II (subtropical and Mediterranean climate)−Long-term storage conditions: 25° C./60±5% relative humidity (RH); test duration: 30 months;

B) ZONE IV.B (hot and very humid climate)—Long-term storage conditions: 30° C. 75±5% RH test duration: 30 months.

An accelerated investigation was also conducted under the following extreme conditions:

C) 40° C./75±5% RH; test duration: 6 months.

TABLE A
25° C./60% RH.

| | T0 | 3 | 6 | 9 | 12 | 18 | 24 | 30 |
|---|---|---|---|---|---|---|---|---|
| Appearance | comp. | comp. | comp. | comp. | comp. | comp. | comp. | comp. |
| Aw | 0.053 | 0.055 | 0.058 | 0.059 | 0.061 | 0.063 | 0.067 | 0.071 |
| CFU (plate count) × | 64.0 | 42.0 | 40.0 | 39.2 | 37.4 | 41.2 | 36.0 | 30.0 |
| 10\hat{ }9/dose (2 g) | | | | | | | | |
| AFU × 10\hat{ }9/dose (2 g) | 86.0 | 72.5 | 73.2 | 83.6 | 84.0 | 83.3 | 70.6 | 76.0 |
| TAMC | comp. | comp. | comp. | comp. | comp. | comp. | comp. | comp. |
| TYMC | comp. | comp. | comp. | comp. | comp. | comp. | comp. | comp. |
| Gram-negative bile- | comp. | comp. | comp. | comp. | comp. | comp. | comp. | comp. |
| tolerant bacteria | | | | | | | | |
| Escherichia coli | comp. | comp. | comp. | comp. | comp. | comp. | comp. | comp. |
| Staphylococcus aureus | comp. | comp. | comp. | comp. | comp. | comp. | comp. | comp. |
| Salmonella spp. | comp. | comp. | comp. | comp. | comp. | comp. | comp. | comp. |

The previous experimental count data (CFU and AFU) are reported in the form of a diagram in FIG. 1.

TABLE B
30° C./75% RH.

| | T0 | 3 | 6 | 9 | 12 | 18 | 24 | 30 |
|---|---|---|---|---|---|---|---|---|
| Appearance | comp. | comp. | comp. | comp. | comp. | comp. | comp. | comp. |
| Aw | 0.053 | 0.057 | 0.06 | 0.062 | 0.065 | 0.083 | 0.086 | 0.090 |
| CFU (plate count) × | 64.0 | 40.0 | 36.0 | 33.2 | 27.0 | 24.0 | 7.0 | 1.3 |
| 10\hat{ }9/dose (2 g) | | | | | | | | |
| AFU × 10\hat{ }9/dose (2 g) | 86.0 | 75.0 | 64.4 | 74.5 | 69.8 | 78.0 | 61.2 | 74.0 |
| TAMC | comp. | comp. | comp. | comp. | comp. | comp. | comp. | comp. |
| TYMC | comp. | comp. | comp. | comp. | comp. | comp. | comp. | comp. |
| Gram-negative bile- | comp. | comp. | comp. | comp. | comp. | comp. | comp. | comp. |
| tolerant bacteria | | | | | | | | |
| Escherichia coli | comp. | comp. | comp. | comp. | comp. | comp. | comp. | comp. |
| Staphylococcus aureus | comp. | comp. | comp. | comp. | comp. | comp. | comp. | comp. |
| Salmonella spp. | comp. | comp. | comp. | comp. | comp. | comp. | comp. | comp. |

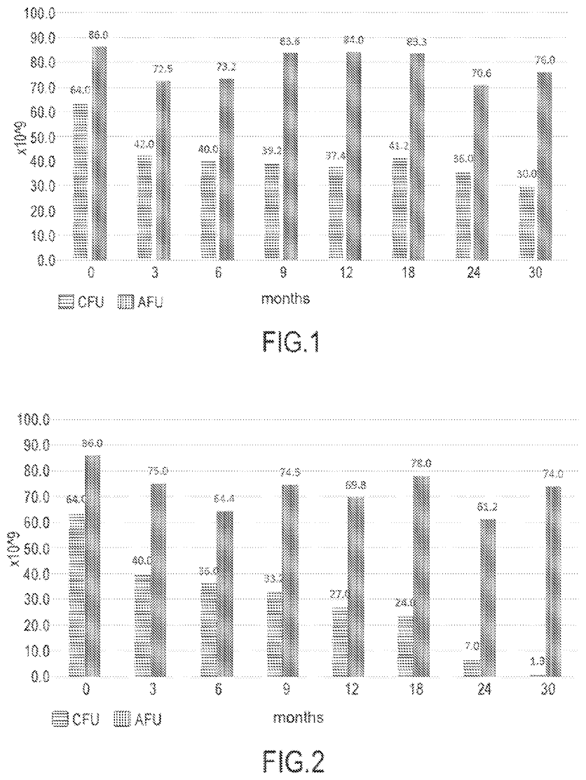

The previous experimental count data (CFU and AFU) are reported in the form of diagram in FIG. 2. FIG. 4 Instead reports the decay rates (k) CFU and AFU as slope values of the slope values of the interpolation line of the experimental data in the Arrhenius linear model, as discussed below.

TABLE C
40° C./75% RH.

| | T0 | 1 | 2 | 3 | 6 |
|---|---|---|---|---|---|
| Appearance | comp. | comp. | comp. | comp. | comp. |
| Aw | 0.053 | 0.057 | 0.063 | 0.069 | 0.075 |
| CFU (plate count) × | 64.0 | 32.6 | 15.0 | 1.4 | 1.6 |
| 10\hat{ }9/dose (2 g) | | | | | |
| AFU × | 86.0 | 57.0 | 43.0 | 33.8 | 42.2 |
| 10\hat{ }9/dose (2 g) | | | | | |
| TAMC | comp. | comp. | comp. | comp. | comp. |
| TYMC | comp. | comp. | comp. | comp. | comp. |
| Gram-negative bile- | comp. | comp. | comp. | comp. | comp. |
| tolerant bacteria | | | | | |
| Escherichia coli | comp. | comp. | comp. | comp. | comp. |
| Staphylococcus aureus | comp. | comp. | comp. | comp. | comp. |
| Salmonella spp. | comp. | comp. | comp. | comp. | comp. |

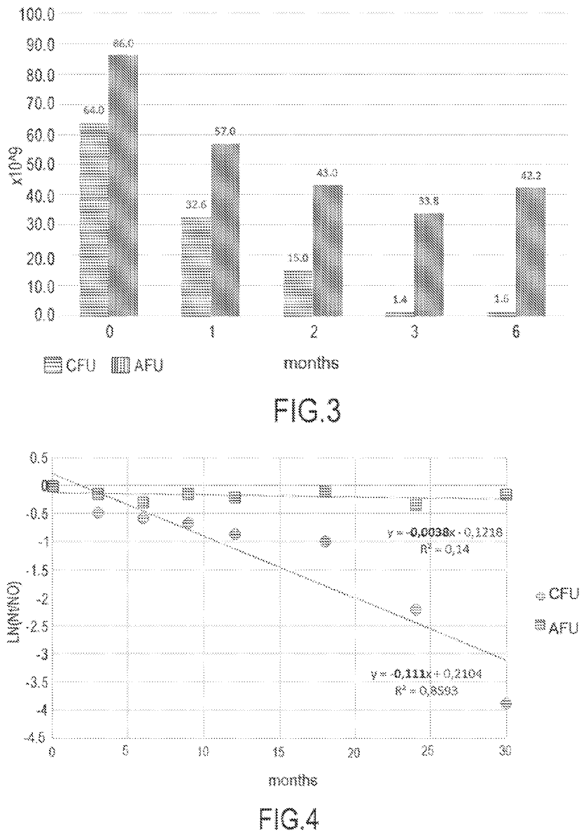

The previous experimental count data (CFU and AFU) are reported in the form of a diagram in FIG. 3.

From the above experimental results it can be observed that the data relating to the chemical-physical and microbiological parameters (appearance, aw, TAMC, TYMC, gram-negative bile-tolerant bacteria, Escherichia coli, Staphyloccocus aureus and Salmonella spp.) have always been found to comply with the rules applicable in all the conditions applied, even the most extreme ones.

Furthermore, it is important to observe the value relative to active fluorescent units (AFU) which—it should be borne in mind—is the index that defines the number of bacterial cells with the integral cell membrane, and therefore still viable.

According to the inventors of the present invention, the AFU values are of extreme importance to fully understand the viability and functionality of the bacterial cells analysed, since the CFU value could be distorted by the presence of viable but not cultivable cells (VBNC).

As a matter of fact, the CFU does not account for dormant or non-colony-generating cells, but which in any case exhibit metabolic activity or which—under suitable environmental conditions (for example at contact with the enteric system)—could recover from sublethal damage.

Furthermore, the integral cells (AFU), regarding which the cultivable cells (CFU) represent a subgroup, can be intended as packets of functional units represented by the bacterial genome; and that therefore the monitoring of a bacterial population in terms of membrane integrity overcomes the requirement of the cultivability and functionality of the cell intended only as the ability to replicate and possibly colonise, but also as a vector of genetic information. This approach therefore opens up to a potential application which is still unexplored since, thanks to the ability of the bacterial cell to transmit genetic information horizontally, it is possible to integrate the gut microbiota with new information transported by the integral cell (AFU).

As regards the Arrhenius model mentioned above, this model was constructed to evaluate the influence of temperature on the stability—by way of example—of Lactobacillus rhamnosus GG (ATCC 53103) Predictive microbiology describes the exponential loss of bacterial viability over time, following a first-order drop, as indicated by the representation of the natural logarithm LN (Nₜ/No) with respect to time (t) as indicated in the equation below.

Nₜ=N₀e^{-kt}

wherein:
Nₜ=bacterial count at time t;
N₀=bacterial count at time zero;
k=decay rate.

From the above equation it is therefore possible to calculate the decimal reduction time (D1), which is defined as the time necessary for the concentration of viable bacterial cells to reach one tenth of the initial amount. For example; the D1 value shown in the tables below shows the values of the decimal reduction time expressed in months, calculated according to the equation:

D1=ln 10/k

The decay rate (k) can be determined for any temperature, based on the slope of each interpolation line. Referring to FIG. 4, the decay rate data are shown in the following table D) for the CFU values and in table E) for the AFU values.

TABLE D
CFU decay rate (plate count),

| | 1/T * | k | | D1 |
|---|---|---|---|---|
| | 1000 (K⁻¹) | (months⁻¹) | LNk | (months) |
| Test A): | 3.354016435 | 0.016 | −4.135166557 | 143.9 |
| 25° C. (298.15 K) | | | | |
| 60% RH | | | | |
| Test B): | 3.298697015 | 0.111 | −2.198225078 | 20.7 |
| 30° C. (303.15 K) | | | | |
| 75% RH | | | | |
| Test C): | 3.193357816 | 0.6616 | −0.413094135 | 3.5 |
| 40° C. (313.15 K) | | | | |
| 75% RH | | | | |

The above table also shows the calculations relating to test A) and to test C), although these data are not shown in a diagram such as that of FIG. 4 for test B).

TABLE E
AFU decay rate (flow cytofluorometry).

| | 1/T * | k | | D1 |
|---|---|---|---|---|
| | 1000 (K⁻¹) | (months⁻¹) | LNk | (months) |
| Test A): | 3.354016435 | 0.0021 | −6.165818 | 1096.5 |
| 25° C. (298.15 K) | | | | |
| 60% RH | | | | |
| Test B): | 3.298697015 | 0.0038 | −5.572754 | 605.9 |
| 30° C. (303.15 K) | | | | |
| 75% RH | | | | |
| Test C): | 3.193357816 | 0.1071 | −2.233992 | 21.5 |
| 40° C. (313.15 K) | | | | |
| 75% RH | | | | |

The above table also shows the calculations relating to test A) and to test C), although these data are not shown in a diagram such as that of FIG. 4 for test B).

The decimal reduction times D1 outlined in table E) show surprising times for decimal reduction of the bacterial cells, which reach more than 21 months in the most drastic preservation conditions. Thus, the stability of the present composition is ensured even in summer periods, and under conditions of uncontrolled increase of air conditioning.

It is also important to note that the AFU parameter allows a faithful photograph of the actual viability of the micro-organisms to be obtained, due to the integrity of the cell membrane of these micro-organisms and in spite of the possible presence of viable but not cultivable cells (VBNC).

### Example 7: Analytical Detection of Potassium Pyrophosphate Ions, Sucrose and Oxygen Free Radicals in the Cryoprotection Solution According to Example 2A

Tests were carried out to identify pyrophosphate ions, sucrose and oxygen free radicals in the cryoprotection solution, carried out according to the indications of the assays reported in the European Pharmacopoeia.

The analytical evaluations were carried out on a set of 6 liquid samples containing the cryoprotection solution prepared according to Example 2A.

Samples were taken in duplicate, at different times, as reported below:
sample 1: solution prepared according to Example 2A
sample 2: solution prepared according to Example 2A pasteuised (sample taken on the day of pasteurisation)
sample 3: solution prepared according to example 2A at 1 day from pasteurisation
sample 4: solution prepared according to Example 2A at 3 days from pasteurisation
sample 5: solution prepared according to Example 2A at 5 days from pasteurisation
sample 6: solution prepared according to Example 2A at 7 days from pasteurisation

The qualitative analysis for the evaluation of the presence of pyrophosphate and sucrose in the samples was carried out following the indications of the assays reported in the European Pharmacopoeia.

Pyrophosphate Analysis

In particular, 5 ml of a silver nitrate solution are added to 5 ml of a solution containing pyrophosphate, neutralised if necessary. As a consequence, a white precipitate is formed.

Sucrose Analysis

0.15 ml of a fresh prepared copper sulphate solution and 2 ml of diluted sodium hydroxide solution are added to 5 ml of a solution containing sucrose. The solution becomes blue and transparent and it does not change after boiling. 4 ml of diluted hydrochloric acid are added to the hot solution and the mixture is brought to a boil for 1 minute. 4 ml of a diluted sodium hydroxide solution are added. An orange precipitate is formed.

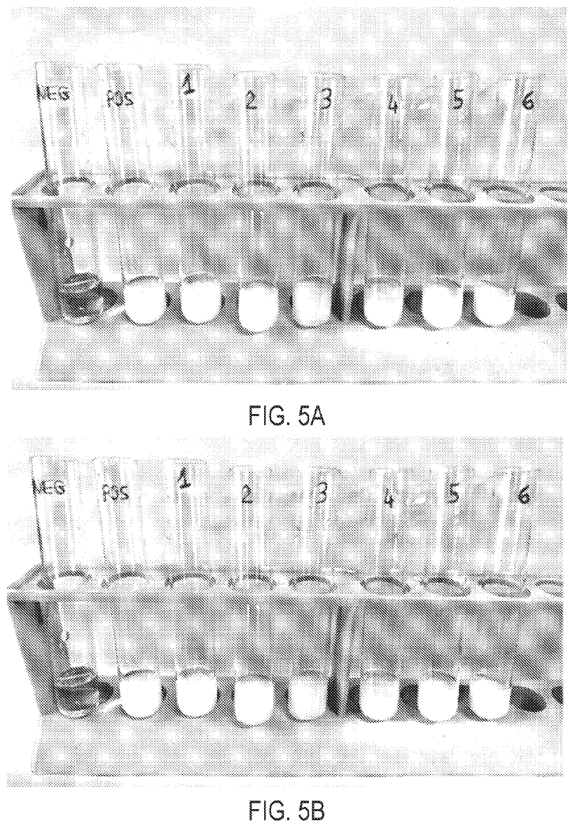

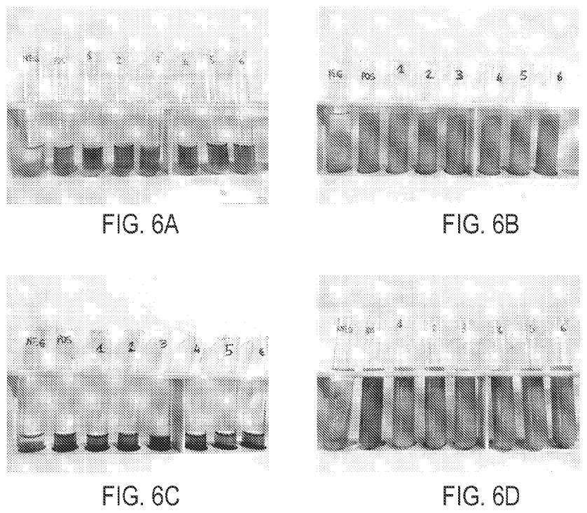

The aforementioned detection assays were conducted on the two sets of six cryoprotection solution samples according to Example 2A and on a known Potassium Pyrophosphate solution and on a known Sucrose solution, used as a positive control in the respective analyses. Distilled water was used as a negative control (see FIG. 5 and FIG. 6).

The Pyrophosphate analysis showed that all the liquid samples analysed in the first and second set (corresponding to the duplicate sampling) meet the Potassium Pyrophosphate detection assay requirements because the white precipitate which characterises the presence of the substance in solution is present (FIG. 5).

The sucrose analysis showed that all liquid samples analysed in the first and second set (corresponding to duplicate sampling) meet the sucrose detection assay requirements as demonstrated by the initial formation of a blue solution, followed by precipitation of an orange solid (FIG. 6).

Subsequently, the chemical structure of pyrophosphate and sucrose was detected by means of infrared spectroscopy.

The six liquid samples and the corresponding duplicates were evaluated by means of ATR-FTIR infrared spectroscopy using the Perkin Elmer Spectrum 100 FT-IR instrument. The spectral data were acquired by means of software version 10.03.

The FTIR spectrum of Pyrophosphate has characteristic bands. In the region between 1250 and 900 cm−1.

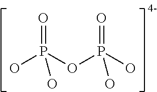

Chemical Structure of Pyrophosphate.

In particular, the band at about 900 cm−1 regards the vibrational stretching of the P—O—P group.

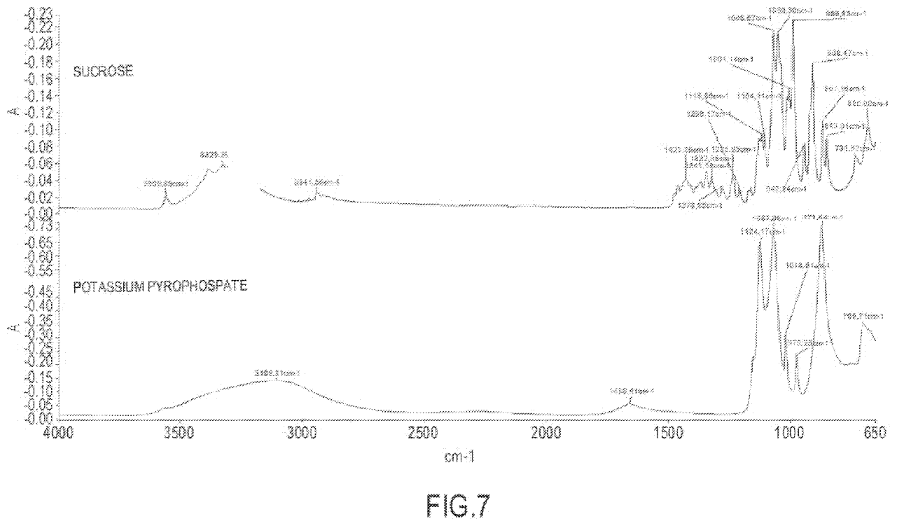

Peaks at about 1018 cm−1 at 973 cm−1 can be detected in the powdered Potassium Pyrophosphate sample analysed as a reference for the evaluation of the set of liquid samples (see FIG. 7).

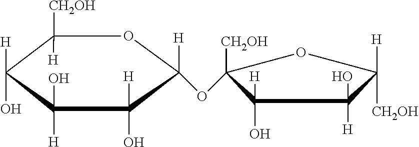

Chemical Structure of Sucrose

The FTIR spectrum of powdered Sucrose shows—in the region comprised between about 3500 and 3325 cm−1—the characteristic peaks of the hydroxyl functional groups (OH) referred to both the glucose molecule (at about 3384 cm−1) and the fructose molecule (at about 3327 cm−1). Furthermore, in the spectrum between 1500 and 750 cm−1 there is a set of intense peaks relating to the stretching of the functional groups CO and CC present in the Sucrose molecule.

The characteristic peaks of the substance can be detected in the powdered Sucrose sample analysed as a reference for the evaluation of the set of liquid samples (see FIG. 7).

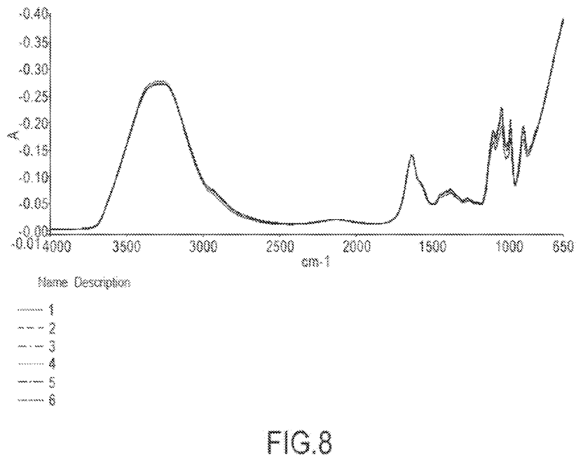

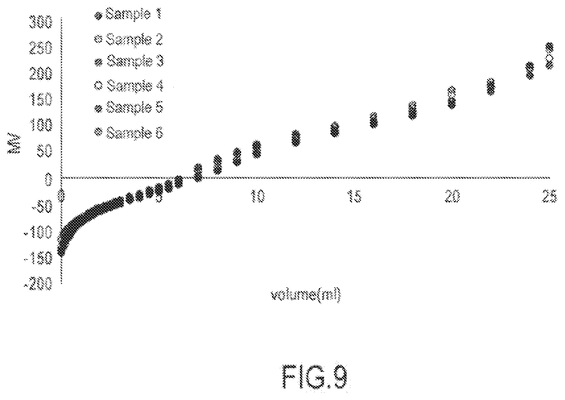

The ATR-FTIR analysis allowed the liquid samples of the two sets under examination to be subjected to FTIR analysis directly. As shown in FIG. 8, the spectra of the analysed samples are all superimposable and they have the absorption bands of Potassium Pyrophosphate and Sucrose.

Subsequently, a quantitative analysis of the potassium pyrophosphate content was carried out in the 6 samples taken in duplicate, by means of potentiometric titration carried out according to the assay reported in European Pharmacopoeia.

For the quantitative determination of the Pyrophosphate content in the liquid samples a potentiometric titration was carried out on 25 ml of the sample using a 1M aqueous solution of HCl.

The volume added at the first inflection point (in mL) is considered for the calculation. As reported in Pharmacopoeia, 1 ml of an aqueous solution of HCl 1M is equivalent to 223.0 mg of Na₄O₇P₂10H₂O.

As shown in FIG. 9, all the curves have a similar profile, and no significant differences in Potassium Pyrophosphate content in the set of 6 liquid sample are observed. The same result was obtained with the corresponding duplicate samples.

In all the samples an inflection point was detected following the addition of a solution volume of HCl 1M comprised between 1 and 1.2 mL.

According to the calculation reported by the Pharmacopoeia reported below. 1 ml of an aqueous solution of HCl 1M is equivalent to 223.0 mg of Na₄O₇P₂,10H₂O.

According to such calculation, the concentration of Potassium Pyrophosphate in the set of liquid samples analysed varies in the range comprised between 12.05 and 14.45 mg/mL.

Subsequently, the quantitative analysis of the sucrose content (g/ml) was carried out by means of high-performance liquid chromatography (HPLC) analysis.

The analytical determination of the amount of sucrose present in the six samples was carried out by high-performance liquid chromatography (HPLC) analysis a reverse phase method.

The parameters used are shown below.

HPLC analysis parameters:
Column: BIO-RAD Bio-Sil NH2 250×4.6 mm
Mobile phase: Acetonitrile-H2O solution (75-25 v/v)
Flow: 1 mL/min.
Detector: refractive index
Total time. 12 minutes
Retention time: approx. 8 minutes

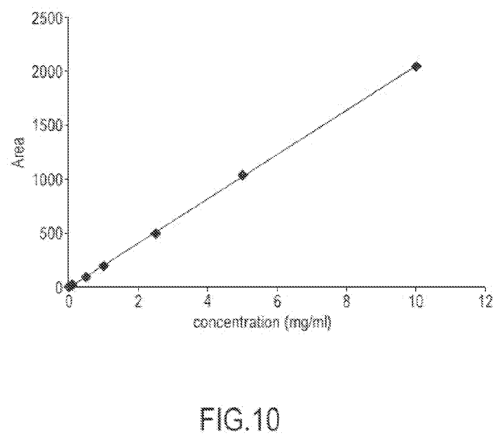

For the calibration curve, a known amount of sucrose was weighed on the analytical scale and dissolved in distilled water. This solution was diluted in the mobile phase to obtain a series of standard solutions in the concentration range comprised between 0.5 and 10 mg/ml. These solutions were injected into HPLC. A linear calibration curve was obtained in the 0.1-10 mg/ml concentration range, having a value of R² of 0.999, see FIG. 10.

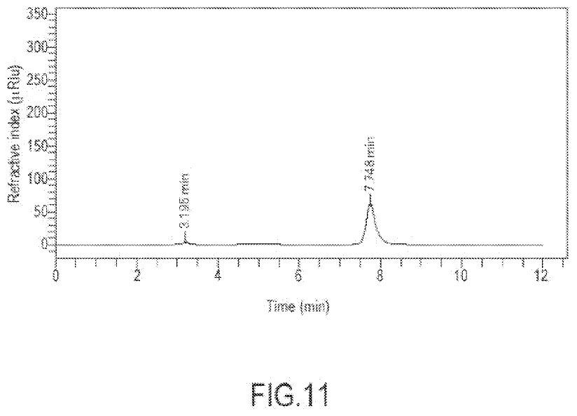

FIG. 11 shows a standard chromatogram of sucrose at the concentration of 5 mg/ml.

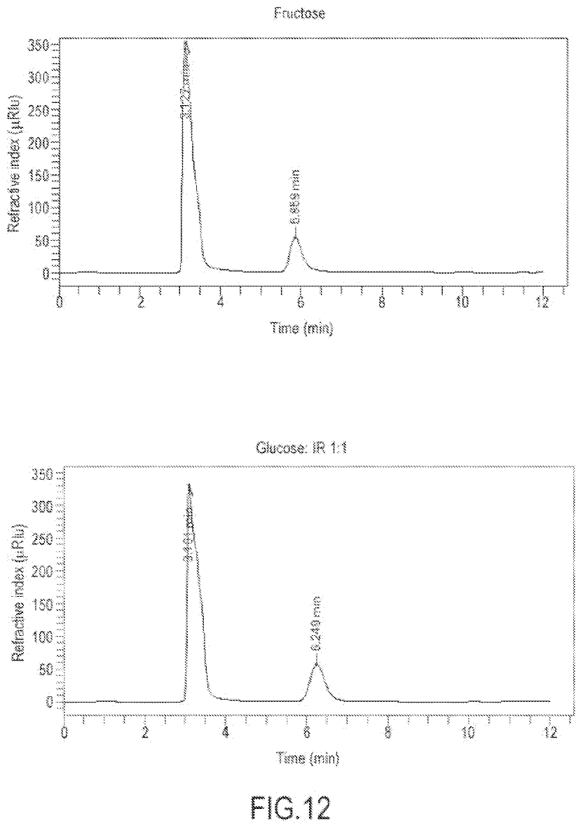

Furthermore, a glucose solution and a fructose solution at the concentration of 5 mg/nl were prepared in distilled water and analysed by means of HPLC under the same analytical conditions. This allows to verify the possible hydrolysis of sucrose in the two monosaccharides, glucose and fructose (FIG. 12).

Given that the chromatographic peaks of glucose and fructose have a different retention time compared to sucrose, it is possible to evaluate the possible presence of hydrolysis.

For the analytical determination of the amount of sucrose present in the six samples (and in the corresponding duplicates), each sample was diluted with a mobile phase volume (1:100 v/v).

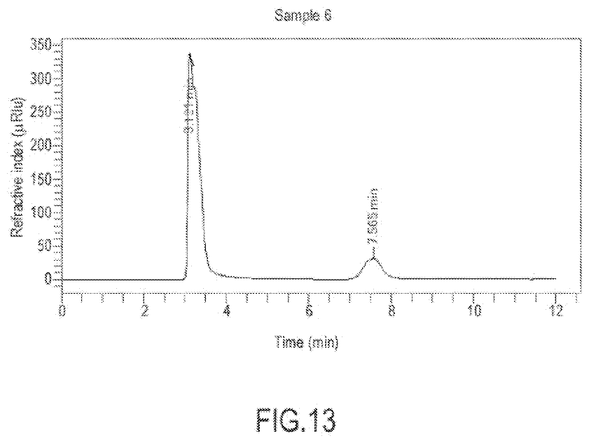

Subsequently each sample was filtered and injected into HPLC. The chromatogram of the Sample 6 is reported in FIG. 13, as an example.

HPLC analysis detected the presence of sucrose in all six samples and in the duplicates while no trace of glucose or fructose was detected. These results demonstrate that sucrose in the samples analysed did not have a hydrolysis process during preservation.

Table 2 reports the concentrations of sucrose measured in the samples by means of HPLC analysis.

TABLE 2
Concentration of DCF in solution and
corresponding absorbance values.

| Sample | Concentration (g/ml) |
|---|---|
| 1 | 0.44 ± 0.03 |
| 2 | 0.41 ± 0.02 |
| 3 | 0.39 ± 0.06 |
| 4 | 0.45 ± 0.03 |
| 5 | 0.45 ± 0.01 |
| 6 | 0.48 ± 0.03 |

The measurements on the individual samples were repeated three times and the value reported in Table 2 is the mean value accompanied by standard deviation.

The analyses were also repeated on the second set of samples, and the values obtained are consistent with those reported for the first set of six samples.

From the results obtained in Example 7, it is possible to conclude that all the liquid samples analysed (after dissolution, pasteurisation and at 1, 3, 5 and 7 days of preservation at refrigerated temperature) meet the assay requirements for the detection of Potassium Pyrophosphate and sucrose. ATR-FTIR spectra are superimposable and potentiometric curves have a similar profile without significant changes in the potassium pyrophosphate content.

Subsequently, the presence of reactive oxygen species (ROS) in the six liquid samples was determined.

To carry out this determination, a fluorimetric method based on the oxidation of the fluorescent probe H₂DCFDA (2′,7′-dichlorodihydrofluorescein diacetate) was used. This molecule does not exhibit fluorescence before being oxidised by the ROS and it is very sensitive to oxidation. This oxidation allows the transformation thereof into fluorescent compound. To this end, 0.5 ml of an ethanoic solution of H2DCFDA (10 mM) 2 ml of NaOH 0.01 M are added to hydrolyse compound H2DCFDA in compound DCFH (non-fluorescent compound). The hydrolysis product is kept at room temperature for 30 minutes and neutralised with 10 ml of PBS phosphate buffer (50 mM, pH 7.2). In the presence of ROS the DCFH compound is rapidly oxidised to DCF (2′, 7′-dichlorofluorescein).

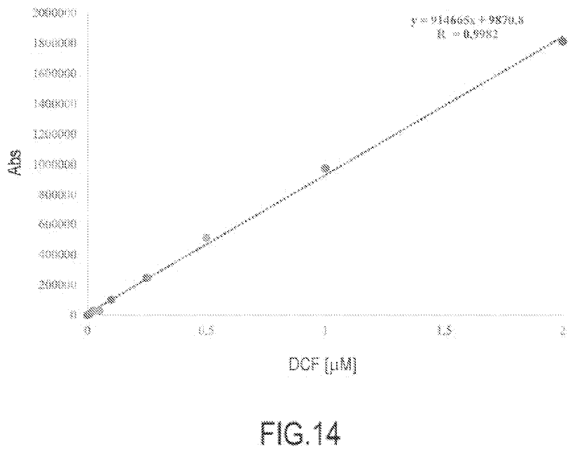

The green fluorescence of the DCF compound was measured using a spectrofluorometer (EnSight™ automated multimode plate reader instrument, Perkin Elmer) set at an excitation wavelength equal to 485 nm and an emission wavelength equal to 530 nm). The concentration of ROS was determined using a calibration curve constructed by measuring the fluorescence of a set of standard DCF solutions, in the concentration range comprised between 0.001-2 μm (FIG. 14).

From the measurements a linear calibration curve was obtained in the concentration range comprised between 0.001-2 μm, with a value of R² of 0.998.

In order to determine ROS in the set of 6 samples, liquids and duplicates, they are diluted with distilled water (1:100 v/v). After 2 ml of diluted sample, a solution of DCFH is added at a concentration equal to 5 μm. Samples are left at room temperature away from light for 20 minutes to complete the reaction. The fluorescence intensity present in the samples is then measured with a spectrofluorometer (485 nm excitation, 530 nm emission) over a period of 60 minutes.

In the two sets of samples under examination, the concentrations of ROS reported in Table 3 were determined.

| | | ROS | |
|---|---|---|---|
| | | (μM) | |
| Sample no | Sample | 1^{st} set | 2^{nd} set |
| — | Distilled water | 1.20 ± 0.260 | |
| 1 | solution after | 1.79 ± 0.167 | 1.42 ± 1.116 |
| | dissolving the | | |
| | preparation | | |
| 2 | solution after | 2.78 ± 0.046 | 2.24 ± 0.002 |
| | pasteurisation | | |
| 3 | solution at 1 day | 2.69 ± 0.132 | 2.47 ± 0.106 |
| | from pasteurisation | | |
| 4 | solution at 3 days | 2.20 ± 0.265 | 2.56 ± 0.026 |
| | from pasteurisation | | |
| 5 | solution at 5 days | 2.50 ± 0.137 | 2.68 ± 0.237 |
| | from pasteurisation | | |
| 6 | solution at 7 days | 2.43 ± 0.489 | 3.46 ± 0.014 |
| | from pasteurisation | | |

The analytical assay was repeated three times for each sample delivered.

The assay showed an increase in ROS concentration after the pasteurisation process, while the preservation of samples for 7 days at controlled temperature did not affect the concentration of ROS present in the solution.

The test was repeated, in the presence of the freeze-dried viable bacterial cells and following their reconstitution with water.

This analysis showed that viable bacterial cells have no masking effect in the detection of pyrophosphate ions.

Innovatively, the present invention allows to achieve the pre-set objectives.

More precisely, the present invention provides a process capable of freeze-drying viable bacterial cells in the presence of a cryoprotectant, damaging a small amount of cell membranes of the micro-organisms.

Advantageously, the present invention provides an analytical protocol capable of reliably distinguishing viable but not cultivable cells within the total bacterial cells present.

With respect to the embodiments of the aforementioned method, compositions and product, a man skilled in the art may replace or modify the described characteristics according to the contingencies. These embodiments are also to be considered included in the scope of protection formalsed in the following claims.

Furthermore, it should be observed that any embodiment may be implemented independently from the other embodiments described.

The following embodiments are part of the present invention.

E1. A method for preparing a biomass of freeze-dried bacterial cells, comprising the following steps:

(i) fermenting a previously prepared biomass of bacterial cells (bacterial biomass) comprising at least one strain of bacterial cells to obtain a fermented biomass of bacterial cells (fermented biomass);

(ii) concentrating the fermented biomass obtained from step (i) up to obtaining a concentrated biomass of bacterial cells (concentrated biomass) having a bacterial cell concentration comprised from 1×10⁶ cells/ml of liquid biomass to 1×10¹² cells/ml of liquid biomass;

(iii) mixing the concentrated biomass obtained from step (ii) with a solution comprising, or alternatively, consisting of (a) at least one pyrophosphate ion salt or pyrophosphoric acid, and mixtures thereof, and (b) at least one polyhydroxy substance selected from among the group comprising or, alternatively, consisting of sucrose, fructose, lactose, lactitol, trehalose or mannitol, and mixtures thereof to obtain a biomass of cryoprotected bacterial cells (cryoprotected biomass);

(iv) freeze-drying the cryoprotected biomass obtained from step (iii) to obtain a biomass of freeze-dried bacterial cells (freeze-dried biomass).

E2. The method according to E1, comprising, before step (ii):

(i.a) adjusting a pH value of the re-concentrated biomass obtained from step (i), to a pH value comprised from 6±0.1 to 6.5±0.1, to obtain a fermented biomass with adjusted pH.

E3. The method according to embodiments E1 or E2, comprising before step (01):

(ii.a) washing the concentrated biomass obtained from step (ii) to obtain a washed biomass;

(ii.b) re-concentrating the washed biomass obtained from slop (ii.a) to obtain a re-concentrated biomass:

E4. The method according to E1, comprising, before step (iii):

(ii.a) washing the concentrated biomass obtained from step (ii) to obtain a washed biomass;

(ii.b) re-concentrating the washed biomass obtained from step (ii a) to obtain a re-concentrated biomass;

(ii.c) adjusting a pH value of the re-concentrated biomass obtained from step (ii.b), to a pH value comprised from 5±0.1 to 7±0.1, to obtain a biomass with adjusted pH.

E5. The method according to any one of the preceding claims, wherein said (a) at least one pyrophosphate ion salt or pyrophosphoric acid is potassium pyrophosphate and/or sodium pyrophosphate and mixtures thereof.

E6. The method according to any one of the preceding embodiments wherein the concentrated biomass of step (ii) is mixed with a solution comprising or, alternatively, consisting of at least one pyrophosphate ion salt or pyrophosphoric acid, and mixtures thereof (a), of the at least one polyhydroxy substance (b) and (c) L-cysteine.

E7. The method according to any one of the preceding embodiments, wherein the concentrated biomass of step (ii) is mixed with a solution comprising, or alternatively, consisting of at least one pyrophosphate ion salt, preferably sodium and/or potassium pyrophosphate and mixtures thereof (a), of the at least one polyhydroxy substance, preferably sucrose and/or trehalose and mixtures thereof (b), and optionally (c) L-cysteine.

E8. The method according to any one of the preceding embodiments, wherein the concentrated biomass of step (ii) is mixed with a solution comprising or, alternatively, consisting of at least one pyrophosphate ion salt, preferably sodium and/or potassium pyrophosphate and mixtures thereof (a), of the at least one polyhydroxy substance, preferably sucrose and/or trehalose and mixtures thereof (b), optionally (c) L-cysteine, and at least one citric acid salt (d), preferably said salt being sodium and/or magnesium citrate and mixtures thereof.

E9. The method according to any one of the preceding embodiments, wherein the freeze-dried biomass of step (iv) has a concentration of bacterial cells comprised from 1×10⁸ cells/g to 1×10³ cells/g, preferably a concentration comprised from 1×10⁷ cells/g to 1×10¹² cells/g, even more preferably a concentration comprised from 1×10⁸ cells/g to 1×10¹² cells/g, even more preferably a concentration comprised from 1×10⁹ cells/g to 1×10² cells/g, for each gram of freeze-dried biomass obtained from step (v).

E10. The method according to any one of the preceding embodiments, wherein the freeze-drying of step (iv) comprises, after step (iii), the following steps:

(iv.a) freezing the cryoprotected biomass obtained from step (ii) to obtain a frozen biomass;

(iv.b) subliming the ice of the frozen biomass obtained from step (iv.a) to obtain the freeze-dried biomass.

E11. The method according to preceding embodiment, wherein the sublimation of step (iv.b) comprises:

(iv.b.1) a step for the primary drying of the frozen biomass obtained from step (iv.a), and

(iv.b.2) a subsequent secondary drying or desorption, on the biomass obtained from step (iv.b.1), to obtain the freeze-dried biomass.

E12. The method according to any one of the preceding embodiments, comprising, besides steps (i), (ii), (ii) and (iv), the preferred steps of:

(viii) contacting the fermented biomass obtained from step (i), the concentrated biomass obtained from step (ii, the cryoprotected biomass obtained from step (iii), and/or the freeze-dried biomass obtained from step (iv) with two different fluorescent dyes, so as to obtain a fluorescent fermented biomass, a fluorescent concentrated biomass, a fluorescent cryoprotected biomass and/or a fluorescent freeze-dried biomass;

(ix) subsequently to step (viii), by means of flow cytofluorometry, detecting an amount of bacterial cells with integral cell membranes in the fluorescent fermented biomass, in the fluorescent concentrated biomass, in the fluorescent cryoprotected biomass and/or in the fluorescent freeze-dried biomass.

E13. The method according to the preceding embodiment, wherein said amount is expressed as active fluorescent units or cells (AFU) regarding which the following correlation applies:

TFU=AFU+nAFU

wherein:
TFU (total fluorescent units) are the total fluorescent bacterial units or cells;
nAFU (non-active fluorescent units) are the non-active fluorescent bacterial units or cells, with a damaged cell membrane.

E14. The method according to embodiment E12 or E13, wherein said amount of bacterial cells with whole cell membranes is used for monitoring the process parameters that govern step (i), step (ii), step (iii) and/or step (iv).

E15. The method according to any one of the preceding embodiments, comprising, besides steps (i), (ii), (iii) and (iv), a step (v) subsequent to step (lv) wherein the freeze-dried biomass obtained from step (iv) is crushed to obtain a crushed biomass.

E16. A biomass of freeze-dried bacterial cells obtained through the method according to any one of the preceding embodiments.

E17. The biomass according to preceding embodiment, characterised in that it is in solid form, preferably in granule or powder form.

E18. A pharmaceutical composition, or medical device composition, or a cosmetic use composition, or food supplement composition or food product composition or food for special medical purposes (AFMS) composition comprising the biomass of freeze-dried bacterial cells according to any one of embodiments E16-E17.

E19. A cryoprotection solution comprising or, alternatively, consisting of at least one pyrophosphate ion salt or pyrophosphoric acid, and mixtures thereof (a), of at least one polyhydroxy substance (b) and optionally, (c) L-cysteine.

E20. The cryoprotection solution according to embodiment E19, wherein said at least one pyrophosphate ion salt is sodium and/or potassium pyrophosphate and mixtures thereof, and wherein said polyhydroxy substance is sucrose and/or trehalose and mixtures thereof.

E21. The cryoprotection solution according to embodiment E19 and E20, wherein said solution further comprises (d) a citric acid salt, for example sodium and/or magnesium citrate and mixtures thereof.

E22. Use of the at least one pyrophosphate ion salt or pyrophosphoric acid and mixtures thereof, of the at least one polyhydroxy substance (b) and optionally, (c) L-cysteine for cryoprotecting a biomass of bacterial cells (bacterial biomass).

E23. The use according to embodiment E22 wherein said at least one pyrophosphate ion salt is sodium and/or potassium pyrophosphate and mixtures thereof, and wherein said polyhydroxy substance is sucrose and/or trehalose and mixtures thereof.

E24. The use according to embodiments E22 and E23, wherein said solution further comprises (d) a citric acid salt, for example sodium and/or magnesium citrate and mixtures thereof.

1. A method for preparing a biomass of freeze-dried bacterial cells, comprising the following steps:
fermenting a previously prepared bacterial biomass comprising at least one strain of bacterial cells to obtain a fermented biomass;
adjusting a pH value of the fermented biomass to a pH value ranging from 6±0.1 to 6.5±0.1, to obtain a fermented biomass with adjusted pH;
concentrating the fermented biomass with adjusted pH up to obtaining a concentrated biomass having a bacterial cell concentration ranging from 1×10⁶ cells/ml of liquid biomass to 1×10¹² cells/ml of liquid biomass;
mixing the concentrated biomass with a solution comprising: (a) at least one pyrophosphate ion salt, pyrophosphoric acid, or a mixture thereof, and (b) at least one polyhydroxy substance selected from sucrose, fructose, lactose, lactitol, trehalose or mannitol, and mixtures thereof to obtain a cryoprotected biomass;
freeze-drying the cryoprotected biomass to obtain a biomass of freeze-dried bacterial cells thus forming a freeze-dried biomass.

2. The method according to claim 1, further comprising before the mixing step):
washing the concentrated biomass to obtain a washed biomass;
re-concentrating the washed biomass to obtain a re-concentrated biomass.

3. The method according to claim 1, further comprising, before the mixing step:
washing the concentrated biomass to obtain a washed biomass;
re-concentrating the washed biomass to obtain a re-concentrated biomass;
adjusting a pH value of the re-concentrated biomass, to a pH value ranging from 5±0.1 to 7±0.1, to obtain a re-concentrated biomass with adjusted pH.

4. The method according to claim 1, wherein said (a) at least one pyrophosphate ion salt, pyrophosphoric acid, or a combination thereof is potassium pyrophosphate, sodium pyrophosphate or a mixture thereof.

5. The method according to claim 1, wherein the mixing is performed by mixing the concentrated biomass with a solution comprising, (a) at least one pyrophosphate ion, pyrophosphate acid salt, or a mixture thereof, (b) at least one polyhydroxy substance and (c) L-cysteine.

6. The method according to claim 1, wherein the mixing is performed by mixing the concentrated biomass with a solution comprising, (a) at least one pyrophosphate ion salt, (b) at least one polyhydroxy substance, and optionally (c) L-cysteine.

7. The method according to claim 1, wherein the mixing is performed by mixing the concentrated biomass with a solution comprising (a) at least one pyrophosphate ion salt, (b) at least one polyhydroxy substance, optionally (c) L-cysteine, and (d) at least one citric acid salt.

8. The method according to claim 1, wherein the freeze-dried biomass of the freeze-drying step has a concentration of bacterial cells ranging from 1×10⁶ cells/g to 1×10¹³ cells/g, for each gram of freeze-dried biomass obtained from the freeze-drying step.

9. The method according to claim 1, wherein the freeze-drying is performed by the following steps:
freezing the cryoprotected biomass to obtain a frozen biomass;
subliming the ice of the frozen biomass to obtain the freeze-dried biomass.

10. The method according to claim 9, wherein the subliming comprises:
performing primary drying of the frozen biomass to obtain a primary dried biomass, and
performing a subsequent secondary drying or desorption, on the primary dried biomass, to obtain the freeze-dried biomass.

11. The method according to claim 1, further comprising:
contacting the fermented biomass, the concentrated biomass, the cryoprotected biomass, and/or the freeze-dried biomass with two different fluorescent dyes, to obtain a fluorescent fermented biomass, a fluorescent concentrated biomass, a fluorescent cryoprotected biomass and/or a fluorescent freeze-dried biomass;
detecting by flow cytofluorometry an amount of bacterial cells with integral cell membranes in the fluorescent fermented biomass, in the fluorescent concentrated biomass, in the fluorescent cryoprotected biomass and/or in the fluorescent freeze-dried biomass.

12. The method according to claim 11, wherein said amount is expressed as active fluorescent units or cells (AFU) wherein the following correlation applies:
TFU=AFU+nAFU
wherein:
TFU (total fluorescent units) are the total fluorescent bacterial units or cells;
nAFU (non-active fluorescent units) are the non-active fluorescent bacterial units or cells, with a damaged cell membrane.

13. The method according to claim 11, wherein said amount of bacterial cells with whole cell membranes is used for monitoring the process parameters that govern the fermenting step, the concentrating step, the mixing step and/or the freeze-drying step.

14. The method according to claim 1, further comprising, crushing the freeze-dried biomass to obtain a crushed biomass.

15. A biomass of freeze-dried bacterial cells obtained through the method according to claim 1.

16. The biomass according to claim 15, wherein the biomass is in solid form.

17. A pharmaceutical composition, medical device composition, a cosmetic use composition, food supplement composition food product composition or food for special medical purposes (FSMP) composition comprising the biomass of freeze-dried bacterial cells according to claim 15.

18. A cryoprotection solution comprising (a) at least one pyrophosphate ion salt, pyrophosphoric acid, or a mixture thereof, (b) of at least one polyhydroxy substance and optionally, (c) L-cysteine.

19. The cryoprotection solution according to claim 18, wherein said at least one pyrophosphate ion salt is sodium, or potassium pyrophosphate or a mixture thereof, and wherein said at least one polyhydroxy substance is sucrose, trehalose, or a mixture thereof.

20. The cryoprotection solution according to claim 18, wherein said solution further comprises (d) a citric acid salt.

21. A method comprising
contacting a bacterial biomass with a solution comprising (a) at least one pyrophosphate ion salt or pyrophosphoric acid, or a mixture thereof, (b) at least one polyhydroxy substance and optionally, (c) L-cysteine, for cryoprotecting the bacterial biomass.

22. The method according to claim 21 wherein said at least one pyrophosphate ion salt is sodium pyrophosphate, potassium pyrophosphate or a mixture thereof, and wherein said polyhydroxy substance is sucrose, and/or trehalose or a mixture thereof.

23. The method according to claim 21, wherein said solution further comprises (d) a citric acid salt.
